SEQUENCE LISTING (27 sequences)

SEQ ID NO: 1 - protein, 63 aa, Drosophila melanogaster
WKTNKSESQYTSLEYDFRVTCDLNYYGSGCAKFCRPRDDSFGHSTCSETGEIICLTGWQGDYC

SEQ ID NO: 2 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 3 - protein, 63 aa, Mus musculus
WSQDLHSSGRTDLRYSYRFVCDEHYYGEGCSVFCRPRDDAFGHFTCGDRGEKMCDPGWKGQYC

SEQ ID NO: 4 - protein, 63 aa, Rattus norvegicus
WSQDLHSSGRTDLRYSYRFVCDEHYYGEGCSVFCRPRDDAFGHFTCGERGEKMCDPGWKGQYC

SEQ ID NO: 5 - protein, 63 aa, Mus musculus
WRTDEQNDTLTRLSYSYRVICSDNYYGESCSRLCKKRDDHFGHYECQPDGSLSCLPGWTGKYC

SEQ ID NO: 6 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 7 - protein, 63 aa, Rattus norvegicus
WQTLKQNTGIAHFEYQIRVTCDDHYYGFGCNKFCRPRDDFFGHYACDQNGNKTCMEGWMGPEC

SEQ ID NO: 8 - protein, 63 aa, Mus musculus
WQTLKQNTGIAHFEYQIRVTCDDHYYGFGCNKFCRPRDDFFGHYACDQNGNKTCMEGWMGPDC

SEQ ID NO: 9 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 10 - protein, 63 aa, Gallus gallus
WQTLKHNTGAAHFEYQIRVTCAEHYYGFGCNKFCRPRDDFFTHHTCDQNGNKTCLEGWTGPEC

SEQ ID NO: 11 - protein, 63 aa, Gallus gallus
WKTLQFNGPVANFEVQIRVKCDENYYSALCNKFCGPRDDFVGHYTCDQNGNKACMEGWMGEEC

SEQ ID NO: 12 - protein, 63 aa, Mus musculus
WKSLHFSGHVAHLELQIRVRCDENYYSATCNKFCRPRNDFFGHYTCDQYGNKACMDGWMGKEC

SEQ ID NO: 13 - protein, 63 aa, Homo sapiens; identical to SEQ ID NO: 12

SEQ ID NO: 14 - protein, 63 aa, Rattus norvegicus; identical to SEQ ID NO: 12

SEQ ID NO: 15 - protein, 63 aa, Homo sapiens; identical to SEQ ID NO: 12

SEQ ID NO: 16 - protein, 63 aa, Drosophila melanocephala
WKTLDHIGRNARITYRVRVQCAVTYYNTTCTTFCRPRDDQFGHYACGSEGQKLCLNGWQGVNC

SEQ ID NO: 17 - protein, 723 aa, Homo sapiens [sequence omitted]

SEQ ID NO: 18 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 19 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 20 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 21 - protein, 1238 aa, Homo sapiens [sequence omitted]

SEQ ID NO: 22 - protein, 2556 aa, Homo sapiens
  MOD_RES (891): Variable amino acid
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGXYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 23 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 24 - protein, 43 aa, Artificial Sequence
  Description of Artificial Sequence Synthetic peptide consensus sequence
CXXXXXXXXCXXXCXXXXXXXXXXXCXXXXXXXCXXXXXXXXC

SEQ ID NO: 25 - protein, 43 aa, Artificial Sequence; identical to SEQ ID NO: 24
  Description of Artificial Sequence Synthetic peptide consensus sequence

SEQ ID NO: 26 - protein, 43 aa, Artificial Sequence
  Description of Artificial Sequence Synthetic peptide consensus sequence
CXXXYYXXXCXXXCRPRBDXFGHXXCXXXGXXXCXXGWXGXXC

SEQ ID NO: 27 - protein, 175 aa, Artificial Sequence
  Description of Artificial Sequence Synthetic peptide sequence of EGF-like domain
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX

(19) United States
(12) Patent Application Publication
(10) Pub. No.: US 2006/0140943 A1
(43) Pub. Date: Jun. 29, 2006

(54) Immunotherapy using modulators of notch signalling

(76) Inventors: Brian Robert Champion, Cambridge (GB); Roberto Celeste Ercole Solari, Cambridge (GB); Margaret Jane Dallman, Cambridge (GB); Jonathan Robert Lamb, Cambridge (GB); Gerard Francis Hoyne, Cambridge (GB); Emmanuel Cyrille Pascal Briend, Cambridge (GB)

Correspondence Address: FROMMER LAWRENCE & HAUG, 745 FIFTH AVENUE- 10TH FL., NEW YORK, NY, 10151, US

(21) Appl. No.: 11/071,796

(22) Filed: Mar. 3, 2005

Related U.S. Application Data
(63) Continuation-in-part of application No. PCT/GB03/03874, filed on Sep. 5, 2003.

(30) Foreign Application Priority Data
Sep. 5, 2002 (GB) 0220658.9

(51) Int. Cl. A61K 48/00; A61K 38/17; A61K 39/395

(52) U.S. Cl. 424/144.1; 514/44; 514/12

(57) ABSTRACT

There is provided a use of a modulator of Notch signalling for the preparation of a medicament for treatment of Graft Versus Host Disease (GVHD) and diseases and conditions caused by or associated with transplants such as organ, tissue and/or cell transplants (e.g. bone marrow transplants), wherein the modulator is used to reduce the reactivity of cells of the immune system.

### CROSS REFERENCE TO RELATED APPLICATIONS

This application is a continuation-in-part of International Application No. PCT/GB2003/003874, filed on Sep. 5, 2003, published as WO 2004/022730 on Mar. 18, 2004, and claiming priority to GB Application Serial No. 0220658.9, filed Sep. 5, 2002.

Reference is made to U.S. application Ser. No. 09/310,685, filed May 4, 1999; Ser. No. 09/870,902, filed May 31, 2001; Ser. No. 10/013,310, filed Dec. 7, 2001; Ser. No. 10/147,354, filed May 16, 2002; Ser. No. 10/357,321, filed Feb. 3, 2002; Ser. No. 10/682,230, filed Oct. 9, 2003; Ser. No. 10/720,896, filed Nov. 24, 2003; Ser. Nos. 10/763,362, 10/764,415 and 10/765,727, all filed Jan. 23, 2004; Ser. No. 10/812,144, filed Mar. 29, 2004; Ser. Nos. 10/845,834 and 10/846,989, both filed May 14, 2004; Ser. No. 10/877,563, filed Jun. 25, 2004; Ser. No. 10/899,422, filed Jul. 26, 2004; Ser. No. 10/958,784, filed Oct. 5, 2004; Ser. No. 11/050,328, filed Feb. 3, 2005; and Ser. No. 11/058,066, filed Feb. 14, 2005.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a method of treating or preventing GVHD and diseases and/or conditions related to GVHD. The present invention also relates to a method of treating or preventing diseases and/or conditions related to organ, tissue and cells transplants, and particularly, but not exclusively, bone marrow transplants.

### BACKGROUND OF THE INVENTION

After the kidney, bone marrow is the most frequent transplant. Bone marrow transplantation is used as a therapy for a number of malignant and non-malignant haematological diseases, including leukaemia, lymphoma, aplastic anaemia, thalassemia major and immunodeficiency diseases, especially severe combined immunodeficiency (SCID).

As bone marrow transplants increasingly come from unrelated donors, recipients (or hosts) of the transplants are immunologically suppressed before grafting to avoid transplant rejection by the host's immune system. However, because the donor bone marrow contains immunocompetent cells, the graft itself may reject the host, causing graft-versus-host disease (GVHD).

GVHD affects between 50% and 70% of all bone marrow transplant patients and can also affect other (e.g. organ) transplant patients if immune cells are accidentally or co-incidentally transferred. It develops as donor T-cells recognise alloantigens (self-antigens) on the host cells. The activation and proliferation of these T-cells and the subsequent production of cytokines generate inflammatory reactions in the skin, gastrointestinal tract and liver. If it is severe, GVHD can result in generalised erythroderma of the skin, gastrointestinal haemorrhage and liver failure. GVHD is responsible for 20% of deaths following bone marrow transplant treatment.

Various treatments are used to prevent GVHD. Traditionally, a transplant recipient is placed on a regimen of immunosuppressive drugs (e.g. cyclosporin A and methotrexate) to inhibit a donor cell immune response. Alternatively, the donor bone marrow is treated with anti-T-cell antisera or monoclonal antibodies specific for T-cells before transplantation, thereby depleting the offending T-cells. Although T-cell depletion allows successful engraftment, it results in catastrophically high infection rates (because the host does not receive a functional immune system) and increases the likelihood that the marrow will be rejected. Even with the development of peripheral blood stem cell (PBSC) transplants, the problem of GVHD has persisted. There is therefore clearly a need to improve the currently available methods of treating GVHD and other diseases and conditions associated with or caused by bone marrow transplants and other transplants.

One approach that has been used is partial T-cell depletion. A low level of donor T-cell activity can indeed be beneficial insofar as the donor cells will kill any host T-cells that survive immunosuppression treatment and therefore further reduce the risk of graft rejection. However, this method does not completely eliminate the risk of GVHD, nor does it eliminate the risk of infection due to reduced immunocompetence. Improved approaches are therefore required.

A description of the Notch signalling pathway and conditions affected by it may be found, for example, in our published PCT Applications as follows:

PCT/GB97/03058 (filed on 6 Nov. 1997 and published as WO 98/20142; claiming priority from GB 9623236.8 filed on 7 Nov. 1996, GB 9715674.9 filed on 24 Jul. 1997 and GB 9719350.2 filed on 11 Sep. 1997); PCT/GB99/04233 (filed on 15 Dec. 1999 and published as WO 00/36089; claiming priority from GB 9827604.1 filed on 15 Dec. 1999);

PCT/GB00/04391 (filed on 17 Nov. 2000 and published as WO 0135990; claiming priority from GB 9927328.6 filed on 18 Nov. 1999);

PCT/GB01/03503 (filed on 3 Aug. 2001 and published as WO 02/12890; claiming priority from GB 0019242.7 filed on 4 Aug. 2000);

PCT/GB02/02438 (filed on 24 May 2002 and published as WO 02/096952; claiming priority from GB 0112818.0 filed on 25 May 2001);

PCT/GB02/03381 (filed on 25 Jul. 2002 and published as WO 03/012111; claiming priority from GB 0118155.1 filed on 25 Jul. 2001);

PCT/GB02/03397 (filed on 25 Jul. 2002 and published as WO 03/012441; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002); PCT/GB02/03426 (filed on 25 Jul. 2002 and published as WO 03/011317; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002); PCT/GB02/04390 (filed on 27 Sep. 2002 and published as WO 03/029293; claiming priority from GB 0123379.0 filed on 28 Sep. 2001); PCT/GB02/05137 (filed on 13 Nov. 2002 and published as WO 03/041735; claiming priority from GB 0127267.3 filed on 14 Nov. 2001, PCT/GB02/03426 filed on 25 Jul. 2002, GB 0220849.4 filed on 7 Sep. 2002, GB 0220913.8 filed on 10 Sep. 2002 and PCT/GB02/004390 filed on 27 Sep. 2002); PCT/GB02/05133 (filed on 13 Nov. 2002 and published as WO 03/042246; claiming priority from GB 0127271.5 filed on 14 Nov. 2001 and GB 0220913.8 filed on 10 Sep. 2002).

Each of PCT/GB97/03058 (WO 98/20142), PCT/GB99/04233 (WO 00/36089), PCT/GB00/04391 (WO 0135990), PCT/GB01/03503 (WO 02/12890), PCT/GB02/02438 (WO 02/096952), PCT/GB02/03381 (WO 03/012111), PCT/GB02/03397 (WO 03/012441), PCT/GB02/03426 (WO 03/011317), PCT/GB02/04390 (WO 03/029293), PCT/GB02/05137 (WO 03/041735) and PCT/GB02/05133 (WO 03/042246) is hereby incorporated herein by reference

Reference is made also to Hoyne G. F. et al (1999) Int Arch Allergy Immunol 118:122-124; Hoyne et al. (2000) Immunology 100:281-288; Hoyne G. F. et al (2000) Intl Immunol 12:177-185; Hoyne, G. et al. (2001) Immunological Reviews 182:215-227; each of which is also incorporated herein by reference.

### SUMMARY OF THE INVENTION

In broad terms, we have now found that reducing the reactivity of cells of the immune system using the Notch signalling pathway reduces the risk of GVHD and prevents infection.

Accordingly, the present invention provides, in a first aspect, a use of a modulator of Notch signalling for the preparation of a medicament for treatment of Graft Versus Host Disease (GVHD).

In a preferred embodiment the present invention provides, a use of a modulator of Notch signalling for the preparation of a medicament for treatment of Graft Versus Host Disease (GVHD) in bone marrow transplantation.

By treating GVHD we include prolonging allograft and non-allograft survival. We also include treating and/or preventing diseases and conditions caused by or associated with GVHD.

Diseases and conditions caused by or associated with GVHD include infection associated with immuno-suppression, inflammation (including chronic inflammatory pathologies such as sarcoidosis, chronic inflammatory bowel disease, ulcerative colitis and Crohn's pathology; and vascular inflammatory pathologies such as disseminated intravascular coagulation, artherosclerosis and Kawasaki's pathology), erythroderma of the skin, severe blistering, gastrointestinal haemorrage, fulminant liver failure, jaundice, scleroderma, joint contractures, skin ulcers, erythematous macules, erythema, esophageal dysmotility, fevers, anthema, diarrhoea, vomituration, anoraxia, abdominal pain, hepatopathy, hepatic insufficiency, hair loss and generalised wasting syndrome.

In a second aspect, the present invention provides a use of a modulator of Notch signalling for the preparation of a medicament for treatment of diseases and conditions caused by or associated with organ transplants (such as kidney, heart, lung, liver and pancreas transplants), tissue transplants (such as skin grafts) and cell transplants (such as bone marrow transplants and blood transfusions). The present invention relates particularly to bone marrow transplants.

Diseases and conditions caused by or associated with bone marrow transplants include malignant, haematologic or genetic diseases such as leukaemia (Chronic Myeloid Leukaemia, Acute Myeloid Leukaemia, Chronic Lymphocytic Leukaemia, Acute Lymphocytic Leukaemia and/or myelodyspastic syndrome), aplastic anaemia, thalassemia major, multiple myeloma, immunodeficiency diseases (such as severe combined immunodeficiency—SCID, systemic lupus erythematosus—SLE, rheumatoid arthritis, rheumatoid spondylitis, osteoarthritis, gouty arthritis and other arthritic conditions, thyroidosis, scleroderma, diabetes mellitus, Graves' disease, Beschet's disease, etc.) lymphomas (including Hodgkin's and non-Hodgkin's lymphomas such as malignant lymphomas, e.g. Burkitt's lymphoma or Mycosis fingoides), GVHD and infections associated with immuno-suppression.

The modulator of the present invention may be selected from the group consisting of: an organic compound, a inorganic compound, a peptide or polypeptide, a polynucleotide, an antibody, a fragment of an antibody, a cytokine and a fragment of a cytokine.

In one embodiment, the modulator is the modulator is capable of activating and/or up-regulating Notch signalling. Preferably, the modulator is capable of activating and/or up-regulating the expression and/or activity of at least one Notch ligand such as a Notch ligand or a fragment or analogue thereof which retains the signalling transduction ability of Notch ligand (e.g. a polypeptide from the Delta or Serrate family of proteins), or a polynucleotide sequence which encodes therefor.

In a preferred embodiment, preparation of the medicament according to the present invention comprises:
(i) isolating an antigen presenting cell (APC) from a transplant patient;
(ii) exposing the cell to a modulator of Notch signalling; and
(iii) incubating said cell with APCs or lymphocytes from a transplant donor.

“Exposing” means bringing together in such a way that the cell may interact with and/or be modified by the modulator. It therefore includes both simple incubation of a cell with a solution or composition containing a modulator of Notch signalling and expressing such a modulator in the cell itself (e.g. by genetic modification).

Thus, step (ii) may comprises bringing the APC from a transplant patient into direct contact with the modulator; or it may comprise transforming the APC from a transplant patient with the modulator or a polynucleotide sequence encoding the modulator.

Advantageously, the APC of step (i) is a dendritic cell (DC) and the APCs or lymphocytes of step (iii) are T-cells.

In an alternative embodiment of the present invention, the modulator is capable of activating and/or upregulating the expression and/or activity of Notch. Such a modulator will preferably be a Notch ligand (e.g. a polypeptide from the Delta or Serrate family of proteins) or a fragment or analogue thereof which retains the signalling transduction ability of Notch ligand, or a polynucleotide sequence which encodes therefor.

Alternatively, the modulator may be the Notch receptor or a derivative, fragment, variant or homologue thereof, or a polynucleotide sequence encoding therefor. In a preferred embodiment, the modulator will be a constitutively active form of Notch.

Use of such a modulator for the preparation of a medicament comprises:
(i) isolating an APC or lymphocyte from a transplant donor;
(ii) exposing the APC or lymphocyte to the modulator; and
(iii) incubating said cell with APCs from a transplant patient.

Step (ii) may comprise bringing the APC or lymphocyte from a transplant donor into direct contact with the modulator; or transforming the APC or lymphocyte from a transplant donor with the modulator or a polynucleotide sequence encoding the modulator, thereby causing activation and/or up-regulation of the expression and/or activity of Notch in the APC or lymphocyte.

Preferably, the APC or lymphocyte of step (i) is a T-cell and the APCs of step (iii) are dendritic cells (DCs).

In one embodiment of the present invention both Notch and Notch ligand may be activated and/or upregulated.

In a third aspect of the invention, there is provided a method of preparing donor cells for use in a transplant comprising:
(i) isolating an antigen presenting cell (APC) from a transplant patient;
(ii) exposing the cell to a modulator of Notch signalling; and
(iii) incubating said cell with APCs or lymphocytes from the transplant donor.

There is also proved, in a fourth aspect, a method of preparing donor cells for use in a transplant comprising:
(i) isolating an APC or lymphocyte from a transplant donor;
(ii) exposing the APC or lymphocyte to a modulator of Notch signalling; and
(iii) incubating said cell with APCs from a transplant patient.

The modulator and APCs are preferably as defined above and the method is preferably for use in the preparation of donor cells for use in an organ transplants (such as kidney, heart, lung, liver or pancreas transplants), tissue transplants (such as skin grafts) or cell transplants (such as a bone marrow transplants or blood transfusions); although they may of course be used in any transplant where there is a risk of immune cell transfer from the donor to an immuno-compromised patient.

In a fifth aspect of the present invention, there is provided a donor cell prepared according to the method of the invention.

In a sixth aspect, there is provided the use of a donor cell according to the invention for the preparation of a medicament for treatment of GVHD and diseases and conditions caused by or associated with transplants such as organ transplants (e.g. kidney, heart, lung, liver or pancreas transplants), tissue transplants (e.g. skin grafts), or cell transplants (e.g. bone marrow transplants or blood transfusions). Preferably, there is provided the use of a donor cell according to the invention for the preparation of a medicament for treatment of diseases and conditions caused by or associated with bone marrow transplants.

In a seventh aspect, there is provided a pharmaceutical composition for use in the treatment of GVHD and diseases and conditions caused by or associated with transplants such as organ transplants (e.g. kidney, heart, lung, liver or pancreas transplants), tissue transplants (e.g. skin grafts) or cell transplants (e.g. bone marrow transplants or blood transfusions) comprising donor cells according to the invention together with a pharmaceutically acceptable carrier. Preferably, there is provided a pharmaceutical composition for use in the treatment of diseases and conditions caused by or associated with bone marrow transplants.

“Therapy” includes curative, alleviative, prophylactic and diagnostic therapy and the term “therapeutic” shall be construed accordingly. The therapy may be on humans or animals.

Preferably a modulator of Notch signalling will be in a multimerised form.

For example, modulators of Notch signalling in the form of Notch ligand proteins/polypeptides coupled to particulate supports such as beads are described in WO 03/011317 (Lorantis) and in Lorantis' co-pending PCT application PCT/GB2003/001525 (filed on 4 Apr. 2003), the texts of which are hereby incorporated by reference (e.g. see in particular Examples 17, 18, 19 of PCT/GB2003/001525).

Modulators of Notch signalling in the form of Notch ligand proteins/polypeptides coupled to polymer supports are described in Lorantis Ltd's co-pending PCT application No PCT/GB2003/003285 (filed on 1 Aug. 2003 claiming priority from GB 0218068.5), the text of which is herein incorporated by reference (e.g. see in particular Example 5 therein).

### BRIEF DESCRIPTION OF THE DRAWINGS

Various preferred features and embodiments of the present invention will now be described by way of non-limiting example and with reference to the accompanying drawings in which:

FIG. 1 shows aligned amino acid sequences of DSL domains from various Drosophila and mammalian Notch ligands (SEQ ID NOs:1-16);

FIG. 2 shows schematic representations of the Notch ligands Jagged and Delta;

FIGS. 3A-3C show amino acid sequences of human Delta-1 (3A, SEQ ID NO:17), Delta-3 (3B, SEQ ID NO:18) and Delta-4 (3C, SEQ ID NO:19);

FIGS. 4A and 4B show amino acid sequences of human Jagged-1 (4A, SEQ ID NO:20) and Jagged-2 (4B, SEQ ID NO:21);

FIG. 5 shows the amino acid sequence of human Notch1 (SEQ ID NO:22);

FIG. 6 shows the amino acid sequence of human Notch2 (SEQ ID NO:23);

FIG. 7 shows schematic representations of Notch 1-4;

FIG. 8 shows a schematic representation of NotchIC;

FIGS. 9 and 10 show schematic representations of the Notch signalling pathway; and

FIG. 11 shows the results of Example 4.

### DETAILED DESCRIPTION OF THE INVENTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

We have found it possible to provide improved treatment for patients in need of a transplant such as a bone marrow transplant.

Graft Versus Host Disease (GVHD)

MHC antigens are present on all cells. They define the immunological identity of an individual and enable the immune system to distinguish between self and non-self matter. When MHC bearing tissue is transferred from one individual to another via an organ transplant, it is recognised by the T-cells of the recipient as foreign leading to rejection of the tissue in a Host Versus Graft (HVG) reaction. However, when MHC-bearing, immunocompetent cells are transferred from a normal individual to an immunocompromised host (e.g. a bone marrow transplant patient), the grafted immunocompetent cells (mainly T lymphocytes) do not recognise the host MHC complex and initiate a Graft Versus Host reaction leading to GVHD.

GVHD has both an activation (or “afferent”) phase and an effector phase. During the activation phase, T-cells from the donor bone marrow (or other transplant) recognise host peptide-MHC complexes displayed on Antigen Presenting Cells (APCs). Antigen presentation, together with a co-stimulatory signal induces donor T-cell activation and proliferation. Cytokines produced by the activated donor T-cells, including IL-2 (interleukin 2), IFN-g (interferon g) and TNF-a (tumor necrosing factor a), induce the effector phase of GVHD by recruiting and activating a variety of secondary effector cells such as NK cells and macrophages. These cells attack cells of the transplant recipient. The main targets include the skin, gastrointestinal tract, liver and lymphoid organs (Ferrara and Deeg, 1991).

The type of transplant a patient has received, pre-transplant ablative therapy, marrow preparation and concurrent medications can affect the presentation of GVHD. Acute GVHD occurs 10-30 days after transplantation. Chronic GVHD occurs after approximately 100 days post-transplantation. Chronic GVHD usually evolves from acute GVHD but may occur de novo in 20-30% of patients.

Incidence of GVHD is higher in recipients of allogeneic hematopoietic cells than in patients receiving syngeneic or autologous hematopoietic cells. The greatest incidence occurs in patients in whom bone marrow is used as the source of hematopoietic cells.

Peripheral blood stem cells (PBSCs) increasingly are used for autologous grafting. With allogeneic grafting, an increased risk exists of developing chronic GVHD in patients in whom PBSCs are used. This risk may be reduced by the use of cord blood stem cells (CBSCs) which are currently being evaluated as a source for transplantation.

Incidence of GVHD in allogeneic recipients increases with the degree of mismatch of major histocompatibility antigens, but GVHD still occurs in matched donor-recipients regardless of the source of the stem cells (i.e. marrow, PBSCs, CBSCs). Patients receiving autologous hematopoietic cells are at risk of developing GVHD, especially if they receive cyclosporin and/or interferon gamma peritransplants. Patients who develop GVHD after an autologous or syngeneic cell transplant tend to develop a milder form of the disease.

GVHD has also been reported after solid organ transplant (especially liver) and after transfer of immunocompetent maternal cells to a relatively immunosuppressed foetal recipient.

Acute GVHD consists of tender erythematous macules that may coalesce over time. Acute GVHD is observed 10-30 days post-transplant. Eruptions usually begin as faint tender erythematous macules on any body part (palms and soles often present first). When erythematous macules form on the trunk or limbs, erythema has been noted to form preferentially around the hair follicle. As the disease progresses, more erythematous macules form and may coalesce to form confluent erythema. The erythematous macules may evolve into papules. In the most severe cases, subepidermal bullae form, and the disease resembles toxic epidermal necrolysis. A staging system for the skin involvement in acute GVHD has been outlined:
Stage 1—Less than 25% body surface involvement
Stage 2—25-50% body surface involvement
Stage 3—50-100% body surface involvement (erythroderma)
Stage 4—Vesicles and bullae
Some patients develop stage 1 GVHD that responds to therapy and never progresses further. Other patients develop a fulminant form that quickly evolves from erythroderma to a lichen planus-like eruption. Patients who develop acute GVHD may also develop massive gastrointestinal bleeding or fulminant liver failure and jaundice.

Patients with chronic GVHD exhibit skin changes that resemble either lichen planus or scleroderma, sometimes simultaneously or sequentially. Chronic GVHD evolves from acute GVHD in 70-90% of patients. The risk of developing chronic GVHD increases with the severity of the acute GVHD syndromes (e.g. patients with stage 3 or stage 4 acute GVHD are more likely to develop chronic GVHD than patients with stage 1 or stage 2 acute GVHD).

As erythema subsides in acute GVHD, violaceous lichenified papules arise that are indistinguishable from lichen planus. Typical lacy white patches on the buccal mucosa of lichen planus are often present. Lichenoid papules have a predilection for flexural surfaces. Sclerodermatous changes are seen in patients with chronic GVHD and some patients exhibit scattered sclerodermatous plaques. Other patients develop widespread disease that results in ulcerations, joint contractures and esophageal dysmotility. The degree of liver and gastrointestinal tract involvement in acute GVHD affects patient outcome. Evidence of liver and/or gastrointestinal tract GVHD without skin involvement is rare. Patients who develop chronic GVHD may also develop skin ulcers, hair loss and a generalised wasting syndrome.

Other major symptoms associated GVHD include frequent fever, anthema, diarrhoea, vomiturition, anorexia, abdominal pain, hepatopathy and hepatic insufficiency. Patients with acute or chronic GVHD are immuno-suppressed and at risk of life-threatening opportunistic infections similar to those that develop in AIDS patients.

Acute GVHD occurs in approximately 50% of patients who receive bone marrow transplants and is a primary or contributory cause of death in 15-45% of the 50% of the patients who develop GVHD after bone marrow transplant. The post-transplant period is also associated with immune dysfunction due to use of prior ablative radio/chemotherapy to suppress the recipient's lymphoid system (especially mature T lymphocytes). This in turn often results in severe infections, which are also a major cause of morbidity and mortality in transplant patients.

As used herein, the term “GVHD” includes any one or more of the symptoms of the disease so that reference to treatment of GVHD includes treatment of, for example, liver failure and/or scleroderma.

Therapeutic strategy for the treatment of GVHD requires a selective suppression of T-cell alloreactivity together with protection against opportunistic infections. We have now found that Notch can be used to reduce the reactivity of (i.e. to “tolerise”) donor T-cells and therefore lower the risk of GVHD without affecting the immune system's ability to fight infection.

Cell Transplants

1. Bone Marrow Transplants

Bone marrow transplants are used to treat a variety of malignant, haematologic and genetic diseases such as thalassia major, immunodeficiency diseases especially severe combined immunodeficiency (SCID), leukaemia (Chronic Myeloid Leukaemia, Acute Myeloid Leukaemia, Chronic Lymphocytic Leukaemia or Acute Lymphocytic Leukaemia), aplastic anaemia, multiple myeloma, lymphomas and other malignant diseases.

The bone marrow, which is obtained from a living donor by multiple needle aspirations, comprises erythroid, myeloid, monocytoid, megakaryocytic and lymphocytic lineages. The graft, usually about 10⁹ cells per kg of host body weight, is injected intravenously into the recipient.

Application of this therapy is, however, limited by the availability of suitable bone marrow donors who are genetically related to the patient and share the same antigens on the surface of their blood cells. Only 25% of patients have a sibling who is an antigenically matched potential donor (allogenic transplant). Bone marrow transplantation can be offered to those patients who lack an appropriate sibling donor by using bone marrow from antigenically matched, genetically unrelated donors, or by using bone marrow from a genetically related sibling or parent who has no less than three (out of six) matching Major Histocompatibility Complex (MHC) antigens. Non-allogenic transplants, however, increase the risk of graft versus host disease (GVHD) and graft rejection. However, finding a matched donor continues to be a problem.

In the usual procedure, the recipient of a bone marrow transplant is immunologically suppressed before grafting. The immune-suppressed state of the recipient makes graft rejection rare; however, because the donor bone marrow contains immunocompetent cells, the graft may reject the host, causing GVHD.

The present invention seeks to overcome these problems.

2. Blood Cell Transplants and Transfusions

Stem cell transplants, such as Peripheral Blood Stem Cell (PBSC) transplants or Cord Blood Stem Cell (CBSC) transplants, are now used as an alternative to bone marrow transplants. Stem cells (which can be induced to differentiate into any type of blood cell) are isolated from the blood of a donor by apherisis (a filtering process). Stem cells which are induced to differentiate into cells of the immune system such as lymphocytes and, in particular, T-cells, can be used to restore a competent immune system to an immuno-compromised patient. This process does not, however, eliminate the risk of GVHD in patients unable to supply their own stem cells (although the immunologic immaturity of CBSCs may lessen the risk this remains to be tested).

GVHD has also been observed in blood transfusion and maternal foetal transfusion patients. Both procedures may therefore be improved by use of the present invention.

Organ and Tissue Transplants

As mentioned above, the present invention can also be used in the treatment of diseases and conditions caused by or associated with organ or tissue transplants. Indeed, the invention can be used wherever there is a risk of (accidental or co-incidental) immune cell transfer from the donor to an immuno-compromised patient. A brief overview of the most common types of organ and tissue transplants is set out below.

1. Kidney Transplants

Kidneys are the most commonly transplanted organs. Kidneys can be donated by both cadavers and living donors and kidney transplants can be used to treat numerous clinical indications (including diabetes, various types of nephritis and kidney failure). Surgical procedure for kidney transplantation is relatively simple. However, matching blood types and histocompatibility groups is desirable to avoid graft rejection. It is indeed important that a graft is accepted as many patients can become “sensitised” after rejecting a first transplant. Sensitisation results in the formation of antibodies and the activation of cellular mechanisms directed against kidney antigens. Thus, any subsequent graft containing antigens in common with the first is likely to be rejected. As a result, many kidney transplant patients must remain on some form of immunosuppressive treatment for the rest of their lives, giving rise to complications such as infection and metabolic bone disease.

2. Heart Transplantation

Heart transplantation is a very complex and high-risk procedure. Donor hearts must be maintained in such a manner that they will begin beating when they are placed in the recipient and can therefore only be kept viable for a limited period under very specific conditions. They can also only be taken from brain-dead donors. Heart transplants can be used to treat various types of heart disease and/or damage. HLA matching is obviously desirable but often impossible because of the limited supply of hearts and the urgency of the procedure.

3. Lung Transplantation

Lung transplantation is used (either by itself or in combination with heart transplantation) to treat diseases such as cystic fibrosis and acute damage to the lungs (e.g. caused by smoke inhalation). Lungs for use in transplants can only be recovered from brain-dead donors.

4. Pancreas Transplantation

Pancreas transplantation is mainly used to treat diabetes mellitus, a disease caused by malfunction of insulin-producing islet cells in the pancreas. Organs for transplantation can only be recovered from cadavers although it should be noted that transplantation of the complete pancreas is not necessary to restore the function needed to produce insulin in a controlled fashion. Indeed, transplantation of the islet cells alone could be sufficient. Because kidney failure is a frequent complication of advanced diabetes, kidney and pancreas transplants are often carried out simultaneously.

5. Skin Grafting

Most skin transplants are done with autologous tissue. However, in cases of severe burning (for example), grafts of foreign tissue may be required (although it should be noted that these grafts are generally used as biological dressings as the graft will not grow on the host and will have to be replaced at regular intervals). In cases of true allogenic skin grafting, rejection may be prevented by the use of immunosuppressive therapy. However, this leads to an increased risk of infection and is therefore a major drawback in bum victims.

6. Liver Transplantation

Liver transplants are used to treat organ damage caused by viral diseases such as hepititis, or by exposure to harmful chemicals (e.g. by chronic alcoholism). Liver transplants are also used to treat congenital abnormalities. The liver is a large and complicated organ meaning that transplantation initially posed a technical problem. However, most transplants (65%) now survive for more than a year and it has been found that a liver from a single donor may be split and given to two recipients. Although there is a relatively low rate of graft rejection by lung transplant patients, leukocytes within the donor organ together with anti-blood group antibodies can mediate antibody-dependent hemolysis of recipient red blood cells if there is a mismatch of blood groups. In addition, manifestations of GVHD have occurred in liver transplants even when donor and recipient are blood-group compatible.

Notch and Notch Ligands

Notch signalling directs binary cell fate decisions in the embryo. As used herein, the expression “Notch signalling” is synonymous with the expression “the Notch signalling pathway” and refers to any one or more of the upstream or downstream events that result in, or from, (and including) activation of the Notch receptor.

Notch was first described in Drosophila as a transmembrane protein that functions as a receptor for two different ligands, Notch and Serrate. Vertebrates have now been found to express multiple Notch receptors and ligands. At least four Notch receptors (Notch-1, Notch-2, Notch-3 and Notch-4) have been identified to date in human cells (see, for example, GenBank Accession Nos. AF308602, AF308601 and U95299—Homo sapiens).

The Notch protein is described in detail in W002/12890. It consists of an extracellular domain containing up to 36 epidermal growth factor (EGF)-like repeats [Notch 1 and 2=36; Notch 3=34 and Notch 4=29], three cysteine rich repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and, like the ankyrin-like repeats, is involved in binding to a transcription factor, known as Suppressor of Hairless [Su(H)] in Drosophila and CBF1 in vertebrates (Tamura). The Notch receptor present in the plasma membrane comprises a disulphide-linked heterodimer of two Notch proteolytic cleavage products, one comprising an C-terminal fragment consisting of a portion of the extracellular domain, the transmembrane domain and the intracellular domain, and the other comprising the majority of the extracellular domain.

The Notch receptor is activated by binding of ligands to the EGF-like repeats of Notch's extracellular domain. Examples of mammalian Notch ligands include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1, Serrate-2 (WO97/01571, WO96/27610 and WO92/19734), Jagged-1 and Jagged-2 (Genbank Accession No. AF029778—Homo sapiens), Scabrous and LAG-2. Notch ligands are characterised by multiple (3-8) EGF-like repeats in their extracellular domains together with a cysteine-rich DSL (Delta-Serrate Lag2) domain comprising 20 to 22 amino acids at the N-terminus of the protein.

Endogenous Notch ligands have been found to be expressed on the surface of cells of the immune system, such as antigen presenting cells (APCs) and T-lymphocytes, and play an important role in the regulation of tolerance induction (WO-A-98/20142).

It has recently been shown that it is possible to generate a class of regulatory T-cells which are able to transmit antigen-specific tolerance to other T-cells, a process termed infectious tolerance (WO-A-98/20142). The functional activity of these cells can be mimicked by over-expression of a Notch ligand protein on their cell surfaces. In particular, regulatory T-cells can be generated by over-expression of a member of the Delta or Serrate family of Notch ligand proteins. Delta or Serrate expressing T-cells specific to one antigenic epitope are also able to transfer tolerance to T-cells recognising other epitopes on the same or related antigens, a phenomenon termed “epitope spreading”.

The present invention provides a method of reducing the reactivity of (or “tolerising”) the immune cells of a donor to the cells of the recipient suitably by incubating them with recipient APCs which have been contacted with a modulator of Notch signalling.

Modulators of Notch Signalling

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to compounds which stimulate or upregulate, at least to some extent, the normal biological activity of the Notch signalling pathway. Conveniently such compounds may be referred to as upregulators or agonists.

The modulator of the present invention may be an organic compound or other chemical. In one embodiment, the modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The modulator may comprise at least one cyclic group. The cyclic group may be a polycyclic group, such as a non-fused polycyclic group. For some applications, the modulator comprises at least the one of said cyclic groups linked to another hydrocarbyl group.

In a preferred embodiment, the modulator will be an amino acid sequence or a chemical derivative thereof, or a combination thereof. Proteins or polypeptides may be in the form of “mature” proteins or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Polypeptide substances may be purified from mammalian cells, obtained by recombinant expression in suitable host cells or obtained commercially. Alternatively, nucleic acid constructs encoding the polypeptides may be used.

Thus, in another preferred embodiment, the modulator will be a nucleotide sequence (which may be a sense or an anti-sense sequence). The modulator may also be an antibody.

The term “antibody” includes intact molecules as well as fragments thereof which are capable of binding the epitopic determinant. These antibody fragments retain some ability to selectively bind with its antigen or receptor and include, for example:

(i) Fab, the fragment which contains a monovalent antigen-binding fragment of an antibody molecule can be produced by digestion of whole antibody with the enzyme papain to yield an intact light chain and a portion of one heavy chain;

(ii) Fab′, the fragment of an antibody molecule can be obtained by treating whole antibody with pepsin, followed by reduction, to yield an intact light chain and a portion of the heavy chain; two Fab′ fragments are obtained per antibody molecule;

(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;

(iv) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

The modulator of the present invention may be a natural isolated compound or a synthetic compound.

Preferably, the modulator of the present invention is a compound capable of stimulating the Notch signalling pathway.

Preferably the modulator of the Notch signalling pathway is an agent capable of activating a Notch receptor (a “Notch receptor agonist”). Suitably for example the modulator may be a Notch ligand or a biologically active fragment or derivative of a Notch ligand.

The term “Notch ligand” as used herein means an agent capable of interacting with and preferably activating a Notch receptor to cause a biological effect. The term as used herein therefore includes naturally occurring protein ligands (e.g. from Drosophila, verterbrates, mammals) such as Delta and Serrate/Jagged (e.g. mammalian ligands Delta1, Delta 3, Delta4, Jagged1 and Jagged2 and homologues) and their biologically active fragments as well as antibodies to the Notch receptor, as well as peptidomimetics, antibodies and small molecules which have corresponding biological effects to the natural ligands.

The term “mimetic” as used herein, in relation to polypeptides or polynucleotides, includes a compound that possesses at least one of the endogenous functions of the polypeptide or polynucleotide which it mimics.

For example, antibodies generated against the Notch receptor are also described in WO 0020576 (the text of which is also incorporated herein by reference). For example, this document discloses generation of antibodies against the human Notch-1 EGF-like repeats 11 and 12. For example, in particular embodiments, WO 0020576 discloses a monoclonal antibody secreted by a hybridoma designated A6 having the ATCC Accession No. HB12654, a monoclonal antibody secreted by a hybridoma designated Cll having the ATCC Accession No. HB12656 and a monoclonal antibody secreted by a hybridoma designated F3 having the ATCC Accession No. HB12655.

Suitably the modulator of the Notch signalling pathway comprises or codes for a protein or polypeptide comprising a Notch ligand DSL or EGF domain or a fragment, derivative, homologue, analogue or allelic variant thereof.

Preferably the modulator of the Notch signalling pathway comprises or codes for a Notch ligand DSL domain and at least one EGF repeat motif, suitably at least 1 to 20, suitably at least 3 to 15, for example at least about 3 to 8 EGF repeat motifs. Suitably the DSL and EGF sequences are or correspond to mammalian sequences. Preferred sequences include mammalian, preferably human sequences.

Preferably the modulator is an agonist of Notch signalling, and preferably an agonist of the Notch receptor (e.g. an agonist of the Notch1, Notch2, Notch3 and/or Notch4 receptor, preferably being a human Notch receptor). Preferably such an agonist (“activator of Notch”) binds to and activates a Notch receptor, preferably including human Notch recpetors such as human Notch1, Notch2, Notch3 and/or Notch4. Binding to and/or activation of a Notch receptor may be assessed by a variety of techniques known in the art including in vitro binding assays and activity assays for example as described herein.

For example, whether any particular agent activates Notch signalling (e.g. is an activator of Notch or a Notch agonist) may be readily determined by use of any suitable assay, for example by use of a HES-1/CBF-1 reporter assay of the type described in WO03/012441 in the name of Lorantis Ltd (e.g. see Examples 8 and 9 therein). Conversely, antagonist activity may be readily determined for example by monitoring any effect of the agent in reducing signalling by known Notch signalling agonists for example, as described in WO03/012441 or WO 03/041735 in the name of Lorantis Ltd (e.g. see Examples 10,11 and 12) (i.e. in a so-called “antagonist” assay).

The Notch Signalling Pathway

Modulators for Notch signalling activation include molecules which are capable of activating Notch, the Notch signalling pathway or any one or more of the components of the Notch signalling pathway.

The Notch signalling pathway in described in WO02/12890. It includes events leading to the activation of Notch, activation of Notch itself, the downstream events of the Notch signalling pathway, transcriptional regulation of downstream target genes and other non-transcriptional downstream events (e.g. post-translational modification of existing proteins). The Notch signalling pathway will also be understood to include the activation and/or expression of target genes.

A very important component of the Notch signalling pathway is Notch receptor/Notch ligand interaction. Thus, Notch signalling may involve changes in expression, nature, amount or activity of Notch ligands or receptors or their resulting cleavage products. In addition, it may involve changes in expression, nature, amount or activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the pathway may involve changes in expression, nature, amount or activity of DNA binding elements such as transcription factors.

In a preferred embodiment, the modulator of the present invention will be capable of inducing or increasing Notch or Notch ligand expression. Such a molecule may be a nucleic acid sequence capable of inducing or increasing Notch or Notch ligand expression.

In a preferred embodiment, the modulator will be a Notch ligand, or a polynucleotide encoding a Notch ligand.

Notch Ligands

Notch ligands are typically capable of binding to a Notch receptor polypeptide present in the membrane of a variety of mammalian cells, for example hemapoietic stem cells. Particular examples of mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and US 6121045 (Millennium)), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-1 (Genbank Accession No. U73936—Homo sapiens) and Jagged-2 (Genbank Accession No. AF029778—Homo sapiens) and LAG-2. Homology between family members is extensive. For example, human Jagged-2 has 40.6% identity and 58.7% similarity to Serrate.

Further homologues of known mammalian Notch ligands may be identified using standard techniques. By a “homologue” it is meant a gene product that exhibits sequence homology, either amino acid or nucleic acid sequence homology, to any one of the known Notch ligands mentioned above. Typically, a homologue of a known Notch ligand will be at least 20%, preferably at least 30%, identical at the amino acid level to the corresponding known Notch ligand over a sequence of at least 10, preferably at least 20, preferably at least 50, suitably at least 100 amino acids or over the entire length of the Notch ligand. Techniques and software for calculating sequence homology between two or more amino acid or nucleic acid sequences are well known in the art (see for example www.ncbi.nlm.nih.gov and Ausubel et al., Current Protocols in Molecular Biology (1995), John Wiley & Sons, Inc.).

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Suitable homologues will be capable of binding to a Notch receptor. Binding may be assessed by a variety of techniques known in the art including in vitro binding assays. Preferably, suitable homologues will comprise at least one distinctive Notch ligand domain.

Some predicted/potential domain locations for various naturally occurring human Notch ligands (based on amino acid numbering in the precursor proteins) are shown below:

Human Delta 1

| Component | Amino acids | Proposed function/domain |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| Component | Amino acids | Proposed function/domain |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| Component | Amino acids | Proposed function/domain |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| Component | Amino acids | Proposed function/domain |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| Component | Amino acids | Proposed function/domain |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence (SEQ ID NO:24):

Cys Xaa Xaa Xaa Xaa Xaa Xaa Xaa Xaa Cys Xaa Xaa
Xaa Cys Xaa Xaa Xaa Xaa Xaa Xaa Xaa Xaa Xaa Xaa
Xaa Cys Xaa Xaa Xaa Xaa Xaa Xaa Xaa Cys Xaa Xaa
Xaa Xaa Xaa Xaa Xaa Xaa Cys

Preferably the DSL domain may include most or all of the following consensus amino acid sequence (SEQ ID NO:25):

Cys Xaa Xaa Xaa ARO ARO Xaa Xaa Xaa Cys Xaa Xaa
Xaa Cys BAS NOP BAS ACM ACM Xaa ARO NOP ARO Xaa
Xaa Cys Xaa Xaa Xaa NOP Xaa Xaa Xaa Cys Xaa Xaa
NOP ARO Xaa NOP Xaa Xaa Cys

wherein:

ARO is an aromatic amino acid residue, such as tyrosine, phenylalanine, tryptophan or histidine;

NOP is a non-polar amino acid residue such as glycine, alanine, proline, leucine, isoleucine or valine;

BAS is a basic amino acid residue such as arginine or lysine; and

ACM is an acid or amide amino acid residue such as aspartic acid, glutamic acid, asparagine or glutamine.

Preferably the DSL domain may include most or all of the following consensus amino acid sequence (SEQ ID NO:26):

Cys Xaa Xaa Xaa Tyr Tyr Xaa Xaa Xaa Cys Xaa Xaa
Xaa Cys Arg Pro Arg Asx Asp Xaa Phe Gly His Xaa
Xaa Cys Xaa Xaa Xaa Gly Xaa Xaa Xaa Cys Xaa Xaa
Gly Trp Xaa Gly Xaa Xaa Cys

(wherein Xaa may be any amino acid and Asx is either aspartic acid or asparagine).

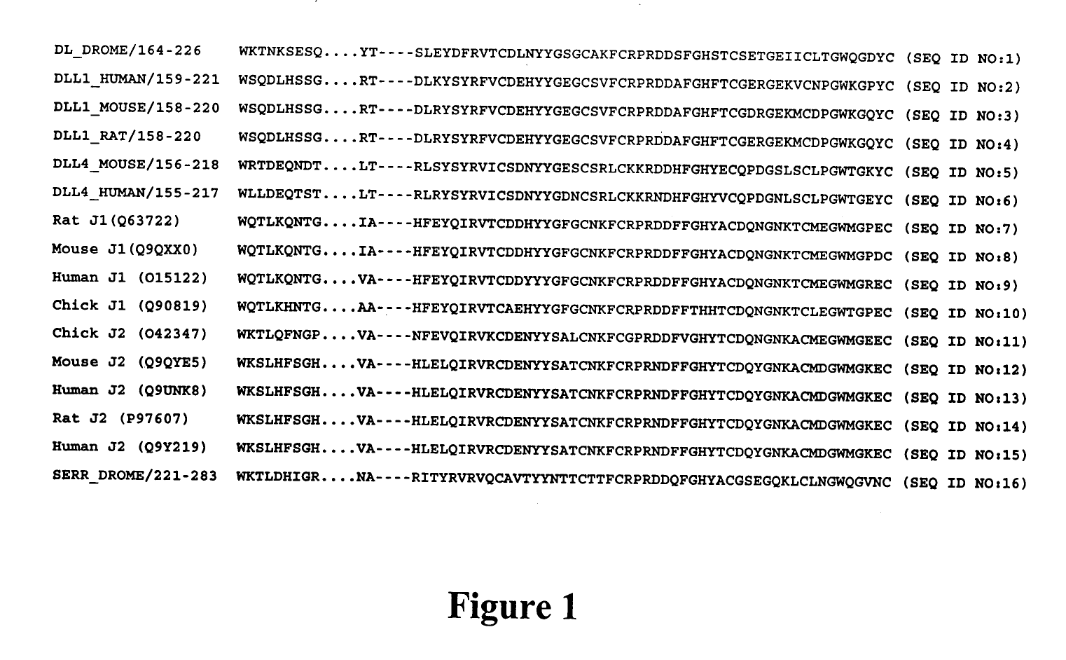

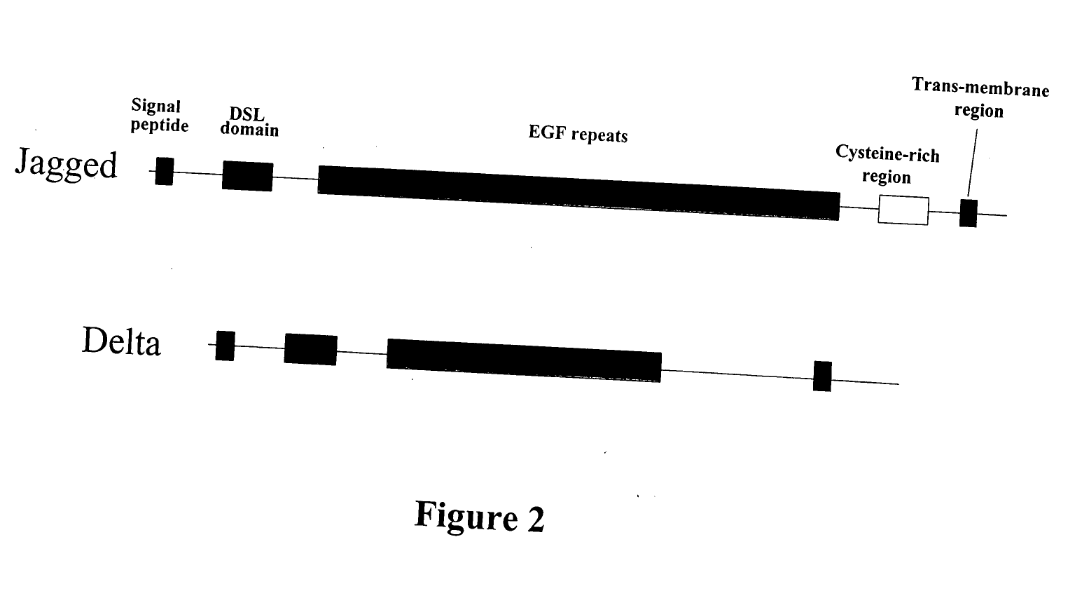

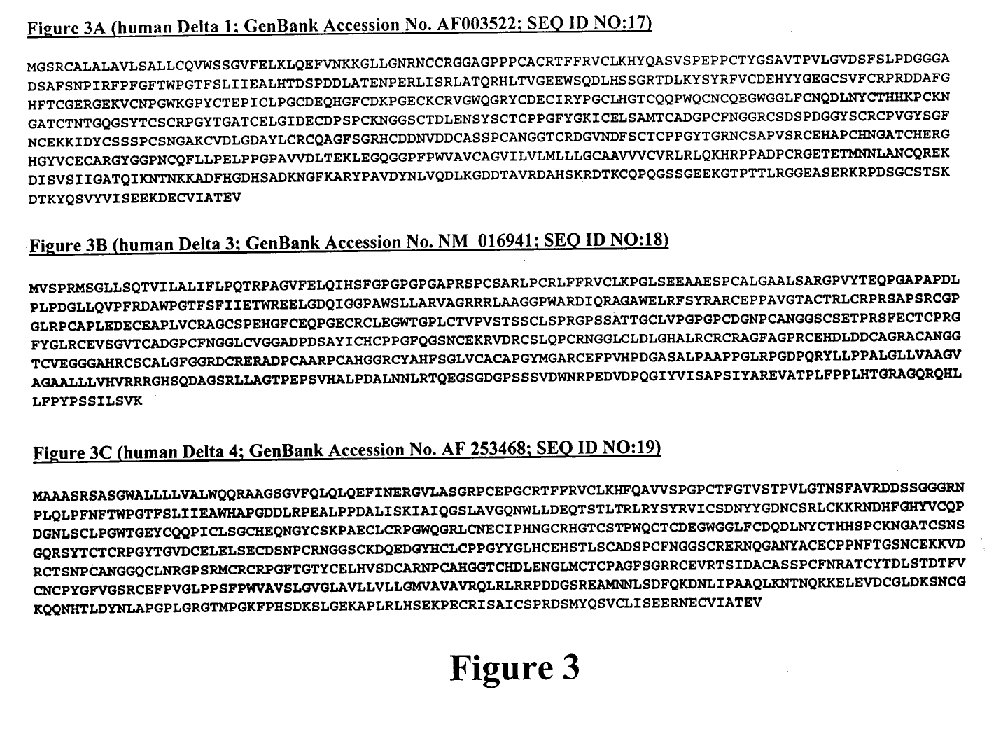

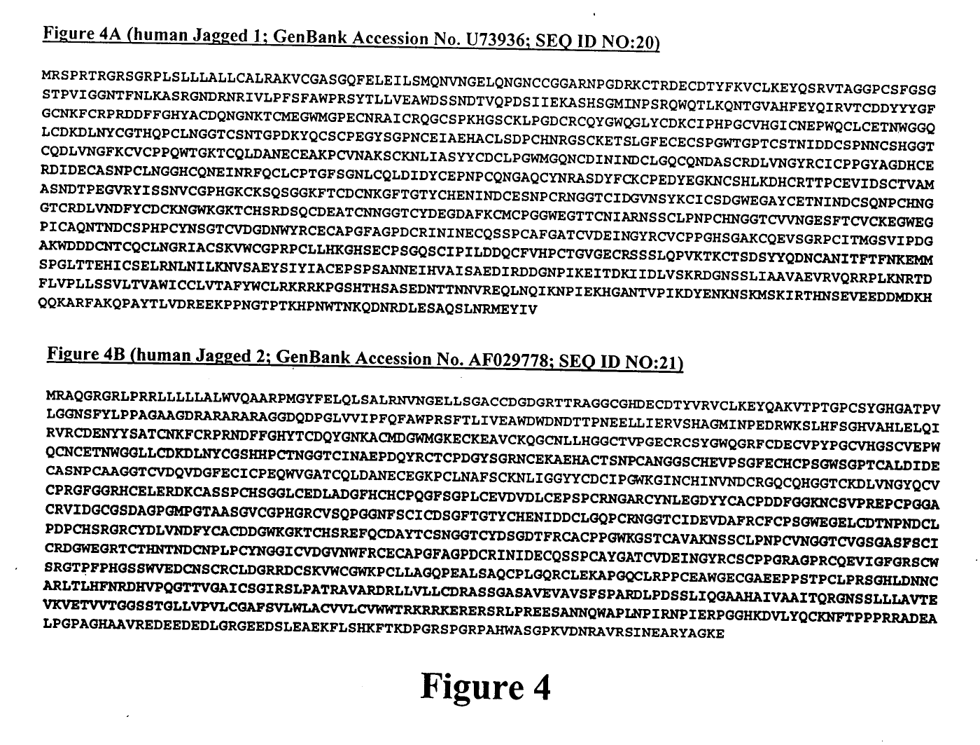

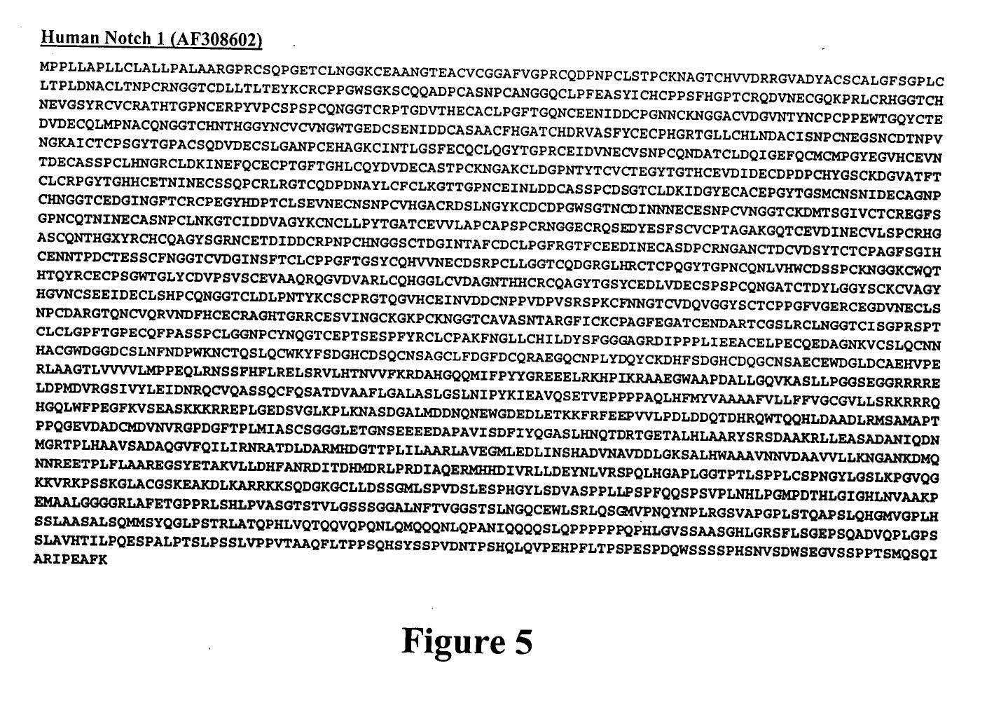

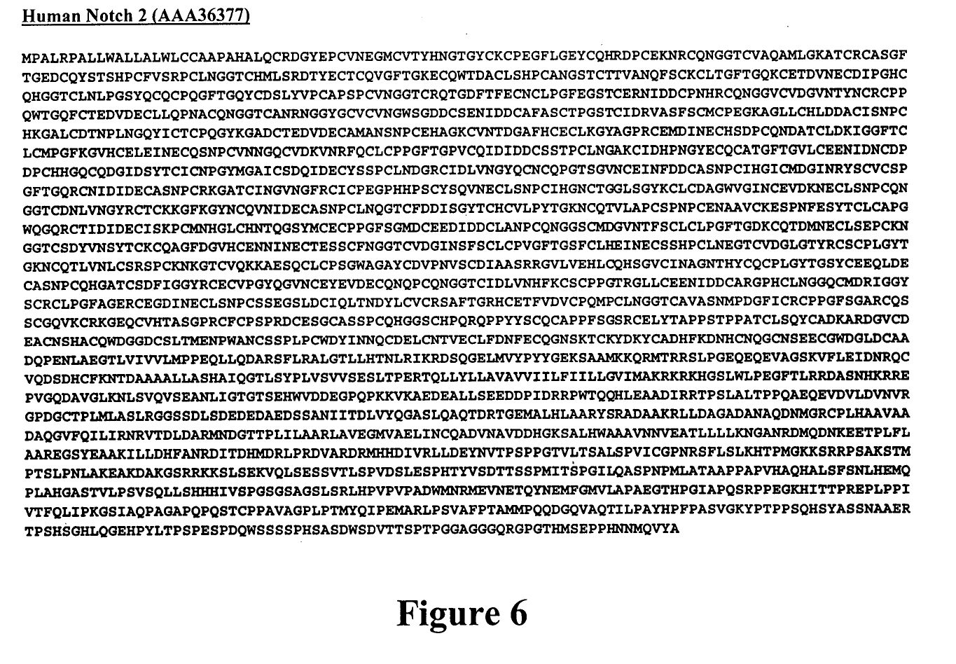

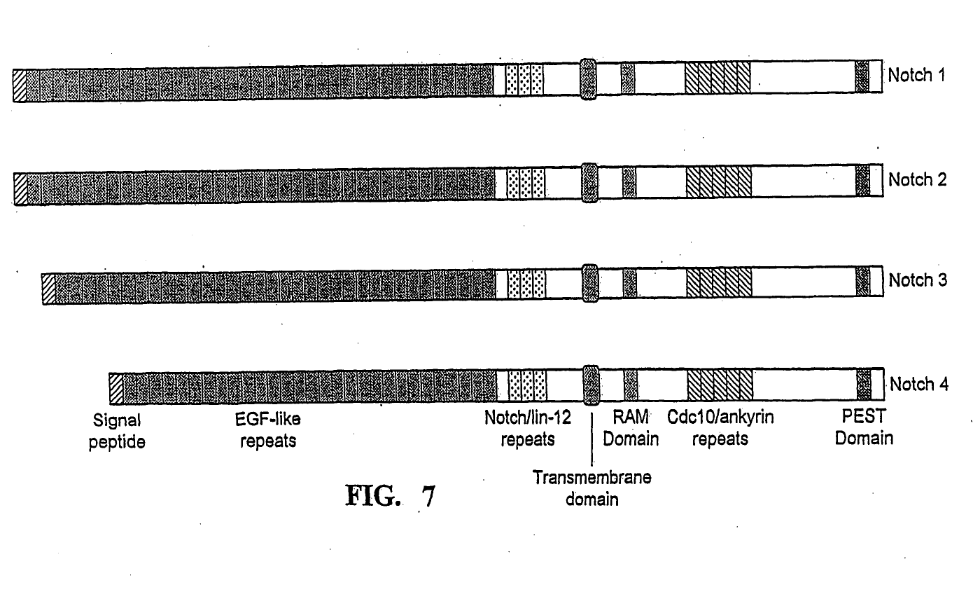

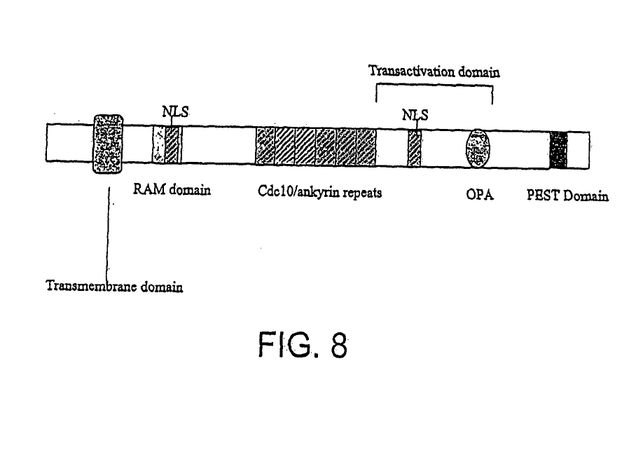

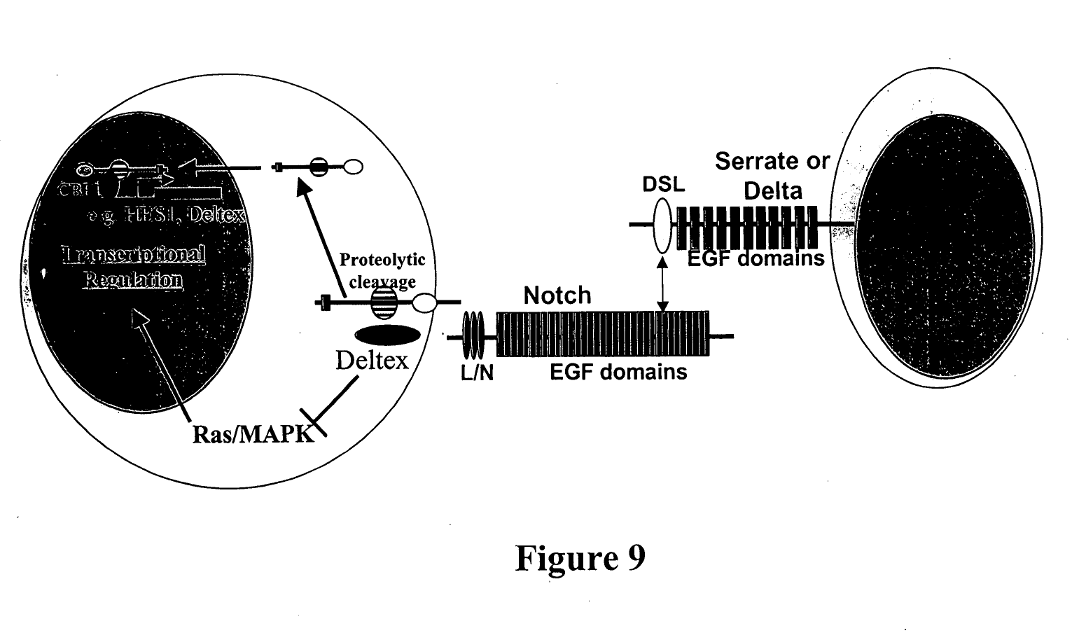

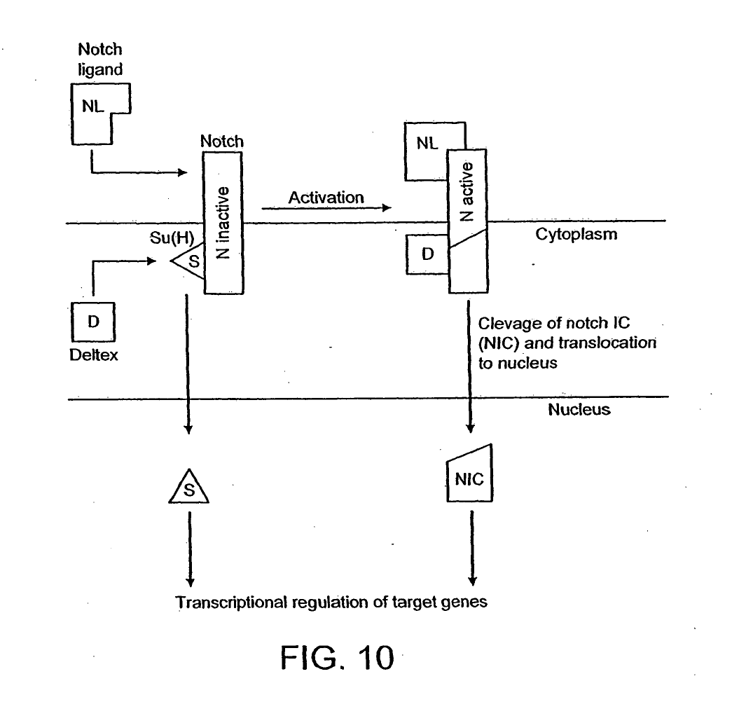

An alignment of DSL domains from Notch ligands from various sources is shown in FIG. 1.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-like Domain

The EGF-like motif has been found in a variety of proteins, as well as Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE the EGF domain typically includes six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of the EGF-like domain (SEQ ID NO:27): 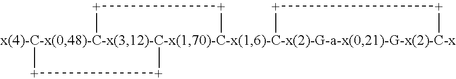
wherein:
‘C’: conserved cysteine involved in a disulfide bond.
‘G’: often conserved glycine
‘a’: often conserved aromatic amino acid
‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

The term “heterologous amino acid sequence” or “heterologous nucleotide sequence” as used herein means a sequence which is not found in the native sequence (e.g. in the case of a Notch ligand sequence is not found in the native Notch ligand sequence) or its coding sequence. Typically, for example, such a sequence may be an IgFc domain or a tag such as a V5His tag.

As a practical matter, the percent identity of any particular amino acid sequence to any another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity. Suitable parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. [Further details on the calculation of homology are set out below].

A modulator of use in the present invention may be any one of the above compounds, fragments, derivatives and homologues thereof or a combination of any two or more of said compounds. Alternatively, the modulator may be a polynucleotide capable of encoding any of the above polypeptides or a compound capable of affecting (preferably stimulating or up-regulating) the expression and/or activity of any one of said polypeptides.

Inhibitors of Notch Signalling Antagonists

Activation of Notch signalling may also be achieved by repressing inhibitors of the Notch signalling pathway. As such, candidate modulators will include molecules capable of repressing any Notch signalling inhibitors. Preferably the molecule will be a polypeptide, or a polynucleotide encoding such a polypeptide, that decreases or interferes with the production or activity of compounds that are capable of producing a decrease in the expression or activity of one or more Notch ligands. In a preferred embodiment, the modulators will be capable of repressing polypeptides of the Toll-like receptor protein family, cytokines such as IL-12, IFN-γ, TNF-α, and growth factors such as BMPs, BMP receptors and activins.

Binding of BMPs (bone morphogenetic proteins, Wilson and Hemmati-Brivanlou, 1997; Hemmati-Brivanlou and Melton, 1997) to their extracellular receptors leads to inhibition of expression of the transcription factors of the achaete/scute complex and therefore to down-regulation of Delta. Thus, any compound that down-regulates BMP expression and/or prevents BMPs from binding to their receptors may be capable of producing an increase in the expression of Notch ligands such as Delta and/or Serrate. Examples of such compounds include BMP anti-sense polynucleotides; BMP mutants or mimetics capable of blocking BMP receptors by irreversibly binding thereto (or nucleic acid sequences encoding such compounds); and proteins (or nucleic acid sequences encoding proteins) such as Noggin (Valenzuela et al., 1995) and Chordin (Sasai et al., 1994). Noggin and Chordin bind to BMPs thereby preventing activation of their signalling cascade. Consequently, increasing Noggin and Chordin levels may lead to an increase in the expression of Notch ligands such as Delta and/or Serrate.

Other examples of polypeptides that down-regulate or inhibit the expression of Delta and/or Serrate include the Toll-like receptor (Medzhitov et al., 1997) and any other receptors linked to the innate immune system (for example CD14, complement receptors, scavenger receptors or defensin proteins), Mesp2 (Takahashi et al., 2000), immune costimulatory molecules (for example CD80, CD86, ICOS, SLAM); accessory molecules that are associated with immune potentiation (for example CD2, LFA-1) and activin (a member of the TGF-b superfamily). Again, any compound that prevents or decreases expression of these proteins can be used to increase the expression of Notch ligands.

Anti-sense constructs designed to reduce or inhibit the expression of down-regulators of Notch ligand expression (exemplified above) may be oligonucleotides such as synthetic single-stranded DNA. However, more preferably, the antisense is an antisense RNA produced in the patient's own cells as a result of introduction of a genetic vector. The vector is responsible for production of antisense RNA of the desired specificity on introduction of the vector into a host cell.

Any of the above listed compounds may be used to increase Notch ligand expression and/or activity either by co-incubation and direct contact between the modulator and the host APC or by transfer of a polynucleotide construct encoding such a modulator into the host APC and expression thereof. Thus primed APC are then incubated with donor T-cells for induction of tolerance.

Alternatively, host APCs may be incubated with donor T-cells in the presence of a Notch receptor agonist. The agonist will mimic Notch receptor stimulation and therefore induce tolerance in the donor immune cells.

Modulators of the Notch Receptor

In one embodiment, the modulator will be a polypeptide derived from any one of the above-described Notch ligands, fragments, derivatives, mimetics or homologues thereof. Preferably, the modulator will be an active fragment of a Notch ligand, for example a Notch ligand EC domain.

In another embodiment, the modulator will be a constitutively active Notch receptor or Notch intracellular domain, or a polynucleotide encoding such a receptor or intracellular domain. Thus, the modulator may be the Notch polypeptide or polynucleotide or a fragment, variant, derivative, mimetic or homologue thereof which retains the signalling transduction ability of Notch or an analogue of Notch which has the signalling transduction ability of Notch. By Notch, we mean Notch-1, Notch-2, Notch-3, Notch-4 and any other Notch homologues or analogues. Analogues of Notch include proteins from the Epstein Barr virus (EBV), such as EBNA2, BARF0 or LMP2A.

In a particularly preferred embodiment the modulator may be the Notch intracellular domain (Notch IC) or a sub-fragment, variant, derivative, mimetic, analogue or homologue thereof. Suitably the Notch sequence will comprise at least a Notch Ankyrin repeat domain and optionally a Notch LNR domain, Notch RAM domain, Notch OPA domain and/or Notch PEST sequence.

An example of a constitutively active form of Notch can be found in the oncogenic variant of human Notch-1 protein known as TAN-1, which has a truncated extracellular domain.

The activating molecule of the present invention may also be a compound capable of modifying Notch-protein expression or presentation on the cell membrane. Agents that enhance the presentation of a fully functional Notch-protein on the target cell surface include matrix metalloproteinases such as the product of the Kuzbanian gene of Drosophila (Dkuz) and other ADAMALYSIN gene family members.

In an alternative embodiment, the modulator of Notch signalling will act downstream of the Notch receptor. Thus, for example, the activator of Notch signalling may be a constitutively active Deltex polypeptide or a polynucleotide encoding such a polypeptide. Other endogenous downstream components of the Notch signalling pathway include Deltex-1, Deltex-2, Deltex-3, Suppressor of Deltex (SuDx), Numb and isoforms thereof, Numb associated Kinase (NAK), Notchless, Dishevelled (Dsh), emb5, Fringe genes (such as Radical, Lunatic and Manic), PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, polypeptides involved in the proteolytic cleavage of Notch such as Presenilin and polypeptides involved in the transcriptional regulation of Notch target genes. Modulators of use in the present invention will therefore include constitutively active forms of any of the above, analogues, homologues, derivatives, variants, mimetics and fragments thereof.

Modulators for Notch signalling activation may also include any polypeptides expressed and/or activated as a result of Notch activation and any polypeptides involved in the expression of such polypeptides, or polynucleotides encoding for such polypeptides.

Such polypeptides include, for example Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

Such polypeptides may also include the intracellular domain of Notch (“Notch IC”). The term “Notch IC” includes the full intracellular domain of Notch or an active portion of this domain. For example, the sequence may be a sequence comprising or coding for at least amino acids 1848 to 2202 of human Notch1 or a sequence having at least 70%, preferably at least 75%, preferably at least 80%, preferably at least 85%, preferably at least 90%, preferably at least 95% amino acid sequence similarity or identity with this sequence. The sequence may also suitably be derived from human Notch2, Notch3 or Notch4.

The Notch intracellular domain, in its endogenous form, has a direct nuclear function (Lieber). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the cdc10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struh1). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

S3 processing occurs only in response to binding of Notch ligands Delta or Serrate/Jagged. The post-translational modification of the nascent Notch receptor in the Golgi (Munro; Ju) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin; Hicks). Although Drosophila has a single Fringe gene, vertebrates are known to express multiple genes (Radical, Manic and Lunatic Fringes) (Irvine).

Notch can also signal in a CBF1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

Deltex, an intracellular docking protein, replaces Su(H) as it leaves its site of interaction with the intracellular tail of Notch. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Further target genes of the Notch signalling pathway include genes of the Hes family (Hes-1 in particular), Enhancer of Split [E(spl)] complex genes, IL-10, CD-23, CD-4 and Dll-1.

Hes-1 (Hairy-enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrf1 gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. IL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. GI1 783344.

Dlx-1 (distalless-1) (McGuiness) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

Polypeptide Sequences

As used herein, the term “polypeptide” is synonymous with the term “amino acid sequence” and/or the term “protein”. In some instances, the term “polypeptide” is synonymous with the term “peptide”.

“Peptide” usually refers to a short amino acid sequence that is 10 to 40 amino acids long, preferably 10 to 35 amino acids.

The term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

The polypeptide sequence may be prepared and isolated from a suitable source, or it may be made synthetically or it may be prepared by use of recombinant DNA techniques.

Polynucleotide Sequences

As used herein, the term “polynucleotide sequence” is synonymous with the term “polynucleotide” and/or the term “nucleotide sequence”.

The polynucleotide sequence may be DNA or RNA of genomic, synthetic or recombinant origin. They may also be cloned by standard techniques. The polynucleotide sequence may be double-stranded or single-stranded whether representing the sense or antisense strand or combinations thereof.

“Polynucleotide” refers to a polymeric form of nucleotides of at least 10 bases in length and up to 1,000 bases or even more. Longer polynucleotide sequences will generally be produced using recombinant means, for example using PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or-human cell, performing a polymerase chain reaction (PCR) under conditions which bring about amplification of the desired region, isolating the amplified fragment (e.g. by purifyng the reaction mixture on an agarose gel) and recovering the amplified DNA. The primers may be designed to contain suitable restriction enzyme recognition sites so that the amplified DNA can be cloned into a suitable cloning vector.

The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Sources of nucleic acid sequences may be ascertained by reference to published literature or databanks such as GenBank. Nucleic acid sequences encoding the desired first or second sequences may be obtained from academic or commercial sources where such sources are willing to provide the material or by synthesising or cloning the appropriate sequence where only the sequence data is available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

The polynucleotide sequence may comprise, for example, a protein-encoding domain, an antisense sequence or a functional motif such as a protein-binding domain and includes variants, derivatives, analogues and fragments thereof. The termn also refers to polypeptides encoded by the nucleotide sequence.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

For recombinant production, host cells can be genetically engineered to incorporate expression systems or polynucleotides of the invention. Introduction of a polynucleotide into the host cell can be effected by methods described in many standard laboratory manuals (e.g. Davis et al and Sambrook et al.) such as transfection, including calcium phosphate transfection and DEAE-dextran mediated transfection, microinjection, cationic lipid-mediated transfection, electroporation, transduction, scrape loading, ballistic introduction and infection. It will be appreciated that such methods can be employed in vitro or in vivo.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate an active conformation when the polypeptide is denatured during isolation and/or purification.

Variants, Derivatives, Analogues, Homologues and Fragments

In addition to the specific polypeptide and polynucleotide sequences mentioned herein, the present invention also encompasses the use of variants, derivatives, analogues, homologues, mimetics and fragments thereof.

In the context of the present invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides, includes any polypeptide or polynucleotide which retains at least one of the functions of the endogenous polypeptide or polynucleotide but generally has a different evolutionary origin thereto.

The term “mimetic” as used herein, in relation to polypeptides or polynucleotides, refers to a chemical compound that possesses at least one of the endogenous functions of the polypeptide or polynucleotide which it mimics.

Typically, amino acid substitutions may be made, for example from 1, 2 or 3 to 10 or 20 substitutions provided that the modified sequence retains the required ability to modulate Notch signalling. Amino acid substitutions may include the use of non-naturally occurring analogues.

Proteins of use in the present invention may also have deletions, insertions, or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | | | |
|---|---|---|---|
| Symbol | 3-letter | Meaning | Codons |

| | | | |
|---|---|---|---|
| A | Ala | Alanine | GCT, GCC, GCA, GCG |
| B | Asp, Asn | Aspartic, | GAT, GAC, AAT, AAC |
| | | Asparagine | |
| C | Cys | Cysteine | TGT, TGC |
| D | Asp | Aspartic | GAT, GAC |
| E | Glu | Glutandc | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, |
| | | | CTA, CTG |
| M | Met | Methionine | ATG |
| N | Asn | Asparagine | AAT, AAC |
| P | Pro | Praline | CCT, CCC, CCA, CCG |
| Q | Gln | Glutamine | CAA, CAG |
| R | Arg | Arginine | CGT, CGC, CGA, CGG, |
| | | | AGA, AGG |
| S | Ser | Serine | TCT, TCC, TCA, TCG, |
| | | | AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, Gln | Glutamic, | GAA, GAG, CAA, CAG |
| | | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleodtide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Polynucleotide variants will preferably comprise codon optimised sequences. Codon optimisation is known in the art as a method of enhancing RNA stability and therefor gene expression. The redundancy of the genetic code means that several different codons may encode the same amino-acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms. Preferably, at least part of the sequence is codon optimised. Even more preferably, the sequence is codon optimised in its entirety.

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (Devereux). Examples of other software than can perform sequence comparisons include, but are not limited to, the BLAST package, FASTA (Atschul) and the GENEWORKS suite of comparison tools. Both BLAST and FASTA are available for offline and online searching. However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be usefuil where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

Further compounds suitable for use as modulators according to the present invention may conveniently be identified using simple assay procedures.

Assays

Assays for detecting modulators of Notch signalling (and, in particular, up-regulators of Notch ligands) from any number of candidate modulators are described below.

The term “candidate modulator” (or “candidate compound”) is used to describe any one or more molecule(s) which may be, or is suspected of being, capable of functioning as a modulator of Notch signalling. Said molecules may for example be organic “small molecules” or polypeptides. Suitably, candidate molecules comprise a plurality of, or a library of such molecules or polypeptides. These molecules may be derived from known modulators. “Derived from” means that the candidate modulator molecules preferably comprise polypeptides which have been fully or partially randomised from a starting sequence which is a known modulator of Notch signalling. Most preferably, candidate molecules comprise polypeptides which are at least 40% homologous, more preferably at least 60% homologous, even more preferably at least 75% homologous or even more, for example 85 %, or 90 %, or even more than 95% homologous to one or more known Notch modulator molecules, using the BLAST algorithm with the parameters as defined herein.

In one embodiment, the present invention provides an assay comprising the steps of:

(a) providing a culture of immune cells;

(b) optionally transfecting said cells with a reporter construct;

(c) optionally transfecting said cells with a Notch gene;

(d) exposing the cells to one or more candidate compound(s) to be tested; and

(e) determining the difference in Notch signalling between cells exposed to the compound(s) to be tested and cells not so exposed.

The assay of the present invention is set up to detect enhancement of Notch signalling in cells of the immune system by candidate modulators. The method comprises mixing cells of the immune system (e.g. T-cells or APCs), where necessary transformed or transfected, etc. with a synthetic reporter gene, in an appropriate buffer, with a sufficient amount of candidate modulator and monitoring Notch signalling, optionally in the presence of a suitable stimulus (such as an antigen). The modulators may be small molecules, proteins, antibodies or other ligands as described above. Amounts or activity of the target gene (also described above) will be measured for each compound tested using standard assay techniques and appropriate controls. Preferably the detected signal is compared with a reference signal and any modulation with respect to the reference signal measured.

The assay may also be run in the presence of a known antagonist of the Notch signalling pathway in order to identify compounds capable of rescuing the Notch signal.

Any one or more of appropriate targets—such as an amino acid sequence and/or nucleotide sequence—may be used for identifying a compound capable of modulating the Notch signalling pathway in cells of the immune system in any of a variety of drug screening techniques. The target employed in such a test may be free in solution, affixed to a solid support, borne on a cell surface, or located intracellularly. The assay of the present invention is a cell based assay.

The assay of the present invention may be a screen, whereby a number of agents are tested. In one aspect, the assay method of the present invention is a high through put screen.

Techniques for drug screening may be based on the method described in Geysen, European Patent No. 0138855, published on Sep. 13, 1984. In summary, large numbers of different small peptide candidate modulators are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators.

This invention also contemplates the use of competitive drug screening assays in which neutralising antibodies capable of binding a target specifically compete with a test compound for binding to a target.

It is expected that the assay methods of the present invention will be suitable for both small and large-scale screening of test compounds as well as in quantitative assays.

Various nucleic acid assays are also known. Any conventional technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

Target gene presence, amplification and/or expression may be measured in a sample directly, for example, by conventional Southern blotting, Northern blotting to quantitate the transcription of target mRNA, dot blotting (DNA or RNA analysis), or in situ hybridisation, using an appropriately labelled probe. Those skilled in the art will readily envisage how these methods may be modified, if desired.

Generation of nucleic acids for analysis from samples generally requires nucleic acid amplification. Many amplification methods rely on an enzymatic chain reaction (such as a polymerase chain reaction, a ligase chain reaction, or a self-sustained sequence replication) or from the replication of all or part of the vector into which it has been cloned. Preferably, the amplification according to the invention is an exponential amplification, as exhibited by for example the polymerase chain reaction.

Many target and signal amplification methods have been described in the literature, for example, general reviews of these methods in Landegren, U., et al., Science 242:229-237 (1988) and Lewis, R., Genetic Engineering News 10:1, 54-55 (1990). These amplification methods may be used in the methods of our invention, and include polymerase chain reaction (PCR), PCR in situ, ligase amplification reaction (LAR), ligase hybridisation, Qbeta bacteriophage replicase, transcription-based amplification system (TAS), genomic amplification with transcript sequencing (GAWTS), nucleic acid sequence-based amplification (NASBA) and in situ hybridisation. Primers suitable for use in various amplification techniques can be prepared according to methods known in the art.

PCR is a nucleic acid amplification method described inter alia in U.S. Pat. Nos. 4,683,195 and 4,683,202. PCR consists of repeated cycles of DNA polymerase generated primer extension reactions. PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached. PCR can be used to amplify any known nucleic acid in a diagnostic context (Mok et al., (1994), Gynaecologic Oncology, 52: 247-252).

In a preferred embodiment, the effect on expression of an endogenous Notch ligand, such as Delta or Serrate, is determined in the presence or absence of a suitable stimulus (such as an antigen) by measuring transcription initiated from the gene encoding the Notch ligand (see, for example WO-A-98/20142) or by quantitative reverse-transcription polymerase chain reaction (RT-PCR). RT-PCR may be performed using a control plasmid with in-built standards for measuring endogenous gene expression with primers specific for Serrate 1, Serrate 2, Delta 1, Delta 2 and/or Delta 3, for example. This construct may be modified as new ligand members are identified.

Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 10⁶ to 10⁹ have been achieved in one hour at 42° C.

Ligation amplification reaction or ligation amplification system uses DNA ligase and four oligonucleotides, two per target strand. This technique is described by Wu, D. Y. and Wallace, R. B. (1989) Genomics 4:560. The oligonucleotides hybridise to adjacent sequences on the target DNA and are joined by the ligase. The reaction is heat denatured and the cycle repeated.

Alternative amplification technology can be exploited in the present invention. For example, rolling circle amplification (Lizardi et al., (1998) Nat Genet 19:225) is an amplification technology available commercially (RCATT™) which is driven by DNA polymnerase and can replicate circular oligonucleotide probes with either linear or geometric kinetics under isothermal conditions.

In the presence of two suitably designed primers, a geometric amplification occurs via DNA strand displacement and hyperbranching to generate 10¹² or more copies of each circle in 1 hour.

If a single primer is used, RCAT generates in a few minutes a linear chain of thousands of tandemly linked DNA copies of a target covalently linked to that target.

A further technique, strand displacement amplification (SDA; Walker et al., (1992) PNAS (USA) 80:392) begins with a specifically defined sequence unique to a specific target. But unlike other techniques which rely on thermal cycling, SDA is an isothermal process that utilises a series of primers, DNA polymerase and a restriction enzyme to exponentially amplify the unique nucleic acid sequence.

SDA comprises both a target generation phase and an exponential amplification phase.

In target generation, double-stranded DNA is heat denatured creating two single-stranded copies. A series of specially manufactured primers combine with DNA polymerase (amplification primers for copying the base sequence and bumper primers for displacing the newly created strands) to form altered targets capable of exponential amplification.

The exponential amplification process begins with altered targets (single-stranded partial DNA strands with restricted enzyme recognition sites) from the target generation phase.

An amplification primer is bound to each strand at its complementary DNA sequence. DNA polymerase then uses the primer to identify a location to extend the primer from its 3′ end, using the altered target as a template for adding individual nucleotides. The extended primer thus forms a double-stranded DNA segment containing a complete restriction enzyme recognition site at each end.

A restriction enzyme is then bound to the double stranded DNA segment at its recognition site. The restriction enzyme dissociates from the recognition site after having cleaved only one strand of the double-sided segment, forming a nick. DNA polymerase recognises the nick and extends the strand from the site, displacing the previously created strand. The recognition site is thus repeatedly nicked and restored by the restriction enzyme and DNA polymerase with continuous displacement of DNA strands containing the target segment.

Each displaced strand is then available to anneal with amplification primers as above. The process continues with repeated nicking, extension and displacement of new DNA strands, resulting in exponential amplification of the original DNA target.

In an alternative embodiment, the present invention provides for the detection of gene expression at the RNA level. Typical assay formats utilising ribonucleic acid hybridisation include nuclear run-on assays, RT-PCR and RNase protection assays (Melton et al., Nuc. Acids Res. 12:7035). Methods for detection which can be employed include radioactive labels, enzyme labels, chemiluminescent labels, fluorescent labels and other suitable labels.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected MRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

PCR technology as described e.g. in section 14 of Sambrook et al., 1989, requires the use of oligonucleotide probes that will hybridise to target nucleic acid sequences. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating ³²P dATP with the Klenow fragment of DNA polymerase in a random priming reaction, as is well known in the art. Oligonucleotides are usually end-labelled with ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na+ at 65-68 ° C. High stringency conditions can be provided, for example, by hybridisation in an aqueous solution containing 6×SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2−0.1×SSC, 0.1 % SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefor easily identifiable. Thus, cell-based screening assays can be designed by constructing cell lines in which the expression of a reporter protein, i.e. an easily assayable protein, such as β-galactosidase, chloramphenicol acetyltransferase (CAT) or luciferase, is dependent on the activation of a Notch. For example, a reporter gene encoding one of the above polypeptides may be placed under the control of an response element which is specifically activated by Notch signalling. Alternative assay formats include assays which directly assess responses in a biological system. If a cell-based assay system is employed, the test compound(s) identified may then be subjected to in vivo testing to determine their effect on Notch signalling pathway.

In general, reporter constructs useful for detecting Notch signalling by expression of a reporter gene may be constructed according to the general teaching of Sambrook et al (1989). Typically, constructs according to the invention comprise a promoter of the gene of interest (i.e. of an endogenous target gene), and a coding sequence encoding the desired reporter constructs, for example of GFP or luciferase. Vectors encoding GFP and luciferase are known in the art and available commercially.

Sorting of cells, based upon detection of expression of target genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym FACS stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (Q˜100%).

FACS can be used to measure target gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefor generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefor assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al. (1989).

In a preferred embodiment, the invention comprises the use of an antisense nucleic acid molecule, complementary to a target MRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In vivo Expression Technology (IVET) (Camilli). IVET identifies target genes up-regulated during say treatment or disease when compared to laboratory culture.

The present invention also provides a method of detection of polypeptides. The advantage of using a protein assay is that Notch ligand expression can be directly measured. Assay techniques that can be used to determine levels of a polypeptide are well known to those skilled in the art. Such assay methods include radioimmunoassays, competitive-binding assays, protein gel assay, Western Blot analysis, antibody sandwich assays, antibody detection, FACS and ELISA assays. For example, polypeptides can be detected by differential mobility on protein gels, or by other size analysis techniques, such as mass spectrometry. The detection means may be sequence-specific. For example, polypeptide or RNA molecules can be developed which specifically recognise polypeptides in vivo or in vitro.

For example, RNA aptamers can be produced by SELEX. SELEX is a method for the in vitro evolution of nucleic acid molecules with highly specific binding to target molecules. It is described, for example, in U.S. Pat. Nos. 5,654,151, 5,503,978, 5,567,588 and 5,270,163, as well as PCT publication WO 96/38579

The invention, in certain embodiments, includes antibodies specifically recognising and binding to polypeptides. Antibodies may be recovered from the serum of immunised animals. Monoclonal antibodies may be prepared from cells from immunised animals in the conventional manner. The antibodies of the invention are useful for identifying cells expressing the genes being monitored.

Antibodies according to the invention may be whole antibodies of natural classes, such as IgE and IgM antibodies, but are preferably IgG antibodies. Moreover, the invention includes antibody fragments, such as Fab, F(ab′)2, Fv and ScFv. Small fragments, such Fv and ScFv, possess advantageous properties for diagnostic and therapeutic applications on account of their small size and consequent superior tissue distribution.

The antibodies may comprise a label. Especially preferred are labels which allow the imaging of the antibody in neural cells in vivo. Such labels may be radioactive labels or radioopaque labels, such as metal particles, which are readily visualisable within tissues. Moreover, they may be fluorescent labels or other labels which are visualisable in tissues and which may be used for cell sorting.

In more detail, antibodies as used herein can be altered antibodies comprising an effector protein such as a label. Especially preferred are labels which allow the imaging of the distribution of the antibody in vivo. Such labels can be radioactive labels or radioopaque labels, such as metal particles, which are readily visualisable within the body of a patient. Moreover, they can be fluorescent labels or other labels which are visualisable on tissue Antibodies as described herein can be produced in cell culture. Recombinant DNA technology can be used to produce the antibodies according to established procedure, in bacterial or preferably mammalian cell culture. The selected cell culture system optionally secretes the antibody product, although antibody products can be isolated from non-secreting cells.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by an enzyme immunoassay, e.g. a sandwich assay or a dot-assay, or a radioimmunoassay. For isolation of the antibodies, the immunoglobulins in the culture supernatants or in the ascitic fluid can be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with the target antigen, or with Protein-A.

The antibody is preferably provided together with means for detecting the antibody, which can be enzymatic, fluorescent, radioisotopic or other means. The antibody and the detection means can be provided for simultaneous, simultaneous separate or sequential use, in a kit.

The antibodies of the invention are assayed for immunospecific binding by any method known in the art. The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al., eds, 1994, Current Protocols in Molecular Biology, Vol. 1, John Wiley & Sons, Inc., New York, which is incorporated by reference herein in its entirety). Exemplary immunoassays are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2,1% Trasylol) supplemented with protein phosphatase and/or protease inhibitors (e.g. EDTA, PMSF, aprotinin, sodium vanadate), adding the antibody of interest to the cell lysate, incubating for a period of time (e.g. 1-4 hours) at 4 ° C., adding protein A and/or protein G sepharose beads to the cell lysate, incubating for about an hour or more at 4 ° C., washing the beads in lysis buffer and resuspending the beads in SDS/sample buffer. The ability of the antibody of interest to immunoprecipitate a particular antigen can be assessed by, e.g. western blot analysis.

Western blot analysis generally comprises preparing protein samples, electrophoresis of the protein samples in a polyacrylamide gel (e.g. 8%-20% SDS-PAGE depending on the molecular weight of the antigen), transferring the protein sample from the polyacrylamide gel to a membrane such as nitrocellulose, PVDF or nylon, blocking the membrane in blocking solution (e.g. PBS with 3% BSA or non-fat milk), washing the membrane in washing buffer (e.g. PBS-Tween 20), exposing the membrane to a primary antibody (the antibody of interest) diluted in blocking buffer, washing the membrane in washing buffer, exposing the membrane to a secondary antibody (which recognises the primary antibody, e.g. an antihuman antibody) conjugated to an enzymatic substrate (e.g. horseradish peroxidase or alkaline phosphatase) or radioactive molecule (e.g. ³²P or ²⁵I) diluted in blocking buffer, washing the membrane in wash buffer, and detecting the presence of the antigen.

ELISAs generally comprise preparing antigen, coating the well of a 96 well microtitre plate with the antigen, adding the antibody of interest conjugated to a detectable compound such as an enzymatic substrate (e.g. horseradish peroxidase or alkaline phosphatase) to the well and incubating for a period of time, and detecting the presence of the antigen. In ELISAs the antibody of interest does not have to be conjugated to a detectable compound; instead, a second antibody (which recognises the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well.

Up-regulated Notch ligand expression or activity can also be monitored using functional assays such as cell adhesion assays. Increased Notch ligand expression leads to increased adhesion between cells expressing Notch and its ligands. Test cells will be exposed to a particular treatment in culture and radiolabelled or flourescein labelled target cells (transfected with Notch/Notch ligand protein) will be overlayed. Cell mixtures will be incubated at 37° C. for 2 hours. Non-adherent cells will be washed away and the level of adherence measured by the level of radioactivity/immunofluorescence at the plate surface.

It is convenient when running assays to immobilise one of more of the reactants, particularly when the reactant is soluble. In the present case it may be convenient to immobilse any one of more of the candidate modulator, Notch ligand, immune cell activator or immune cell costimulus. Immobilisation approaches include covalent immobilsation, such as using amine coupling, surface thiol coupling, ligand thiol coupling and aldehyde coupling, and high affinity capture which relies on high affinity binding of a ligand to an immobilsed capturing molecule. Example of capturing molecules include: streptavidin, anti-mouse Ig antibodies, ligand-specific antibodies, protein A, protein G and Tag-specific capture. In one embodiment, immobilisation is achieved through binding to a support, particularly a particulate support which is preferably in the form of a bead.

For assays involving monitoring or detection of tolerised T-cells for use in clinical applications, the assay will generally involve removal of a sample of treated cells prior to the step of detecting a signal resulting from cleavage of the intracellular domain.

As used herein, the term “sample” refers to a collection of inorganic, organic or biochemical molecules which is either found in nature (e.g. in a biological- or other specimen) or in an artificially-constructed grouping, such as agents which may be found and/or mixed in a laboratory. The biological sample may refer to a whole organism, but more usually to a subset of its tissues, cells or component parts (e.g. body fluids, including but not limited to blood, mucus, saliva and urine).

Using the methods described above, it is possible to detect compounds that affect the expression, processing or activity of Notch ligands. The invention also relates to compounds detectable by these assays methods, and to their use in the methods of the present invention.

Antigen Presenting Cells and T-cells

Antigen-presenting cells (APCs) for use in the present invention may be “professional” antigen presenting cells or may be another cell that may be induced to present antigens to T-cells. Alternatively an APC precursor may be used which differentiates or is activated under the conditions of culture to produce an APC. An APC for use in the ex vivo methods of the invention is typically isolated from the bone marrow or blood of a transplant patient (or from the residual blood in organs intended for transplantation). Preferably the APC or precursor is of human origin.

APCs include dendritic cells (DCs) such as interdigitating DCs or follicular DCs, Langerhans cells, PBMCs, macrophages, B-lymphocytes, T-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells.

The APC or precursor APC may be provided by a cell proliferating in culture such as an established cell line or a primary cell culture. Examples include hybridoma cell lines, L-cells and human fibroblasts such as MRC-5. Preferred cell lines for use in the present invention include Jurkat, H9, CEM and EL4 T-cells; long-term T-cell clones such as human HA1.7 or mouse D10 cells; T-cell hybridomas such as DO11.10 cells; macrophage-like cells such as U937 or THP1 cells; B-cell lines such as EBV-transformed cells such as Raji, A20 and M1 cells.

The APCs or precursors may be modified by the culture conditions or may be genetically modified, for instance by transfection of one or more genes.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba et al., 1992), or from bone marrow, non-adherent CD34⁺ cells can be treated with GM-CSF and TNF-a (Caux et al., 1992). DCs can also be routinely prepared from the peripheral blood of human volunteers, similarly to the method of Sallusto and Lanzavecchia (1994) using purified peripheral blood mononucleocytes (PBMCs) and treating 2 hour adherent cells with GM-CSF and IL-4. If required, these may be depleted of CD19⁺ B cells and CD3⁺, CD2⁺ T-cells using magnetic beads (see Coffin et al., 1998). Culture conditions may include other cytokines such as GM-CSF or IL-4 for the maintenance and, or activity of the dendritic cells or other antigen presenting cells.

Where T-cells are to be used in the ex vivo methods of the invention, the T-cells are typically T lymphocytes isolated from the bone marrow of a transplant patient or from the transplant donor (in the case of cells to be tolerised). T-cells from the donor are obtained by an appropriate method (e.g. as described in U.S. Pat. No. 4,663,058) and may be enriched and/or purified by standard methods including antibody-mediated separation. The T-cells may be used in combination with other immune cells, obtained from the same or a different individual. Alternatively whole blood may be used or leukocyte enriched blood or purified white blood cells as a source of T-cells and other cell types. It is particularly preferred to use helper T-cells (CD4⁺). Alternatively other T-cells such as CD8⁺ cells may be used. It may also be convenient to use cell lines such as T-cell hybridomas, immature T-cells of peripheral or thymic origin and NK-T cells. In a preferred embodiment, the T-cells used in the present invention will be T-cells that can transfer antigen specific suppression to other T-cells.

Thus, it will be understood that the term “antigen presenting cell or the like” are used herein is not intended to be limited to APCs. The skilled man will understand that any vehicle capable of presenting antigens to the T-cell population may be used. For the sake of convenience the term APCs is used to refer to all these. As indicated above, preferred examples of suitable APCs include dendritic cells, T-cells, hybridomas, fibroblasts, lymphomas, macrophages, B cells or synthetic APCs such as lipid membranes.

Donor APCs of use in the present invention will be taken from donor individuals selected from an appropriate category (live related, MHC-matched unrelated or unmatched).

Introduction of Nucleic Acid Sequences into APCs and T-cells

T-cells and APCs as described above are cultured in a suitable culture medium such as DMEM or other defined media, optionally in the presence of fetal calf serum.

Polypeptide substances may be administered to T-cells and/or APCs by introducing nucleic acid constructs/viral vectors encoding the polypeptide into cells under conditions that allow for expression of the polypeptide in the T-cell and/or APC. Similarly, nucleic acid constructs encoding antisense constructs may be introduced into the T-cells and/or APCs by transfection, viral infection or viral transduction.

In a preferred embodiment, nucleotide sequences encoding the enhancers of Notch ligand expression and/or activity will be operably linked to control sequences, including promoters/enhancers and other expression regulation signals.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyrnvate kinase). Tissue-specific promoters specific for lymptocytes, dendritic cells, skin, brain cells and epithelial cells within the eye are particularly preferred, for example the CD2, CD11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

Any of the above promoters may be modified by the addition of further regulatory sequences, for example enhancer sequences. Chimeric promoters may also be used comprising sequence elements from two or more different promoters.

Alternatively (or in addition), the regulatory sequences may be cell specific such that the gene of interest is only expressed in cells of use in the present invention. Such cells include, for example, APCs and T-cells.

The resulting T-cells and/or APCs that comprise nucleic acid constructs capable of up-regulating Notch ligand expression are now ready for use. If required, a small aliquot of cells may be tested for up-regulation of Notch ligand expression as described above. The cells may be prepared for administration to a patient or incubated with T-cells in vitro (ex vivo).

Preparation of Primed APCs and Lymphocvtes

According to one aspect of the invention, host (or recipient) APCs are used to present antigens or allergens to immune cells from the donor. Thus, for example, host APCs may be cultured in a suitable culture medium such as DMEM or other defined media, optionally in the presence of a serum such as fetal calf serum. Optimum cytokine concentrations may be determined by titration. One or more substances capable of up-regulating the Notch signalling pathway (and in particular capable of up-regulating and/or activating Notch-ligand expression or activity) are then typically added to the culture medium together with donor APCs or T-cells. The donor cells may be added before, after or at substantially the same time as the modulator(s).

It may be preferred to prepare primed host APCs first and then incubate them with donor T-cells. For example, once the primed APCs have been prepared, they may be pelleted and washed with PBS before being resuspended in fresh culture medium.

Incubations will typically be for at least 1 hour, preferably at least 3 or 6 hours or, if necessary, for 12 hours or more, in suitable culture medium at 37° C. If required, a small aliquot of cells may be tested for modulated target gene expression as described above. Induction of immunotolerance may be determined by subsequently challenging T-cells with an antigen of interest and measuring IL-2 production compared with control cells not exposed to APCs.

Notch ligand expression may be induced in the host APCs according to one of the following strategies:

a) Gene therapy with a vector expressing a modulator as described above, such as a Notch ligand or an activator or up-regulator of Notch ligand expression (for example, a transcription factor of the achaete/scute complex). The vector may be a viral vector such as an adenovirus, an adeno-associated virus, a retrovirus vector or it may be a plasmid.

b) Alternatively, the host APCs may be incubated with the donor T-cells in the presence of a Notch receptor agonist such as a peptide derived from Notch ligand so mimicking the Notch receptor stimulus and inducing tolerance in the donor T-cells.

If necessary the modulator may be bound to a membrane or support. Suitably a plurality of modulators will be bound to the membrane or support. Such a membrane or support can be selected from those known in the art. In a preferred embodiment, the support is a particulate support matrix. In an even more preferred embodiment, the support is a bead. The bead may be, for example, a magnetic bead (e.g. as available under the trade name “Dynal”) or a polymeric bead such as a Sepharose bead.

T-cells which have been treated according to the above methods may be used to induce immunotolerance in other cells of the immune system.

Tolerisation Assays

Any of the assays described above (see “Assays”) can be adapted to monitor or to detect reduced reactivity and tolerisation in immune cells for use in clinical applications. Such assays will involve, for example, detecting increased Notch-ligand expression or activity in host cells or monitoring Notch cleavage in donor cells. Further methods of monitoring immune cell activity are set out below.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity.

Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32kD Disulfidelinked Early Activation Antigen (EA-1) by 12-0-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Therapeutic Uses

Successfully tolerised donor T-cells prepared by the method of the invention may be used to treat, or to improve the treatment of, diseases and conditions treated by organ, tissue and cell transplants, particularly bone marrow transplants, and diseases and conditions associated with (e.g. caused by or linked to) such transplants or GVHD. “Treatment” includes diagnostic, prophylactic and therapeutic treatment and the expression “to treat” should be construed accordingly.

Diseases and conditions treated by bone marrow transplant include malignant, haematologic or genetic disease such as leukaemia, aplastic anaemia, multiple myeloma and lymphomas, thalassemia major and immunodeficiency diseases.

Leukemias that can be treated include, for example, chronic myeloid leukaemia, acute myeloid leukaemia, chronic lymphocytic leukaemia, acute lymphocytic leukaemia and/or myelodyspastic syndrome.

Lymphomas that can be treated include Hodgkin's and non-Hodgkin's lymphomas, such as malignant lymphomas (Burkitt's lymphoma or Mycosis fungoides).

Immune disorders that can be treated include severe combined immunodeficiency (SCID), systemic lupus erythematosus (SLE), rheumatoid arthritis, rheumatoid spondylitis, osteoarthritis, gouty arthritis and other arthritic conditions, thyroidosis, scleroderma, diabetes mellitus, Graves' disease, Beschet's disease, and the like.

Diseases and conditions treated by organ transplants include common conditions such as diabetes (including diabetes mellitus) and various types of nephritis, kidney failure (such as that caused by end-stage renal disease), urinological problems, toxic hepatitis, hyperlipidemia, cirrhosis, chronic liver disease, lung disease such as emphysema, cystic fibrosis or acute lung damage (such as that caused by smoke inhalation), alpha-1 antitrypsin malfunction, heart conditions (such as protein losing enteropathy) and heart damage, lymphoproliferative disease, Wilson's disease, biliary atresia and various types of cancer. Many other conditions treatable by organ transplantation will be apparent to the skilled practitioner.

Diseases and conditions associated with (e.g. caused by or linked to) organ, tissue and cell (such as bone marrow) transplants include, for example, GVHD and (severe) infection. Diseases and conditions associated with acute GVHD include inflammatory reactions, severe blistering/erythrodermna, erythematous macules, gastrointestinal bleeding, fulminant liver failure and jaundice. Disorders associated with chronic GVHD include scleroderma that results in erythema, esophageal dysmotility, joint contractures and skin ulcers, hair loss and generalised wasting syndrome. Other major symptoms associated with GVHD include frequent fever, anthema, diarrhoea, vomiturition, anorexia, abdominal pain hepatopathy and hepatic insufficiency.

Infection can be characterised by sepsis syndrome, general sepsis, gram-negative sepsis, septic shock, endotoxic shock, toxic shock syndrome, cachexia, circulatory collapse and shock resulting from acute or chronic bacterial infection, acute and chronic parasitic and/or infectious diseases, bacterial, viral or fungal, such as a HIV, AIDS (including symptoms of cachexia, autoimmune disorders, AIDS dementia complex and infections), fever and myalgias due to bacterial or viral infections. Any of the above can be treated and/or prevented according to the method of the present invention.

Inflammatory reactions that can be treated are, for example, chronic inflammatory pathologies and vascular inflammatory pathologies, including chronic inflammatory pathologies such as sarcoidosis, chronic inflammatory bowel disease, ulcerative colitis, and Crohn's pathology and vascular inflammatory pathologies, such as, but not limited to, disseminated intravascular coagulation, atherosclerosis, and Kawasaki's pathology.

As mentioned above, GVHD is responsible for 20% of deaths following bone marrow transplant treatment. It has now surprisingly been found that the use of tolerised T-cells according to the invention results in a 50% decrease in mortality.

Administration

T-cells prepared by the methods of the present invention for use in a organ, tissue or cell transplant such as a bone marrow transplant are typically formulated for administration to patients, in a therapeutically effective amount, with a pharmaceutically acceptable carrier, diluent and/or excipient to produce a pharmaceutical composition.

As used herein, “a therapeutically effective amount” of cells refers to a number of cells which, when administered, is sufficient to induce at least partial tolerance to an alloantigen in donor cells. Preferably, a sufficient number of cells will be administered to cause an increase in allograft survival while causing no side effects or an acceptable level of side effects.

Suitable carriers and diluents are well known in the art. The choice of carriers will be determined in part by the kind and number of cells delivered, as well as by the particular method used to administer the composition. Accordingly, there is a wide variety of suitable formulations for the pharmaceutical composition of the present invention.

Formulations suitable for intravenous and intraperitoneal administration, for example, include aqueous and nonaqueous, isotonic sterile injection solutions (such as isotonic saline solutions), which can contain anti-oxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. Further suitable carriers and diluents are described in Remington's Pharmaceutical Sciences, Mack Publishing Co. (A. R. Gennaro edit. 1985).

The exact amount of such compounds required will vary from subject to subject, depending on the species, age, and general condition of the subject, the severity of the disease that is being treated, its mode of administration (e.g. intravenous, intra-arterial, or peritoneal administration) and the like. The dosage will vary according to such factors as degree of compatibility of the donor and recipient, the health of the host, and the amount of immunosuppressant drugs given concurrently. Thus, it is not possible to specify an exact activity-promoting amount. However, an appropriate amount may be determined by one of ordinary skill in the art using routine testing.

The composition of the present invention may be administered by any suitable means. One skilled in the art will appreciate that many suitable methods of administering the composition to an animal in the context of the present invention, in particular a human, are available and that, although more than one route may be used, a particular route of administration may provide a more immediate and more effective reaction than another.

The composition may be administered by parenteral, subcutaneous, intrapulmonary, and intranasal administration, and if desired for local immunosuppressive treatment, intralesional administration (including perfusing or otherwise contacting the graft with the immunosuppressive agent prior to transplantation). Parenteral infusions include intramuscular, intravenous, intraarterial, or intraperitoneal administration. In addition, the composition is suitably administered by pulse infusion, particularly with declining doses of the composition. Preferably, the dosing is given by injections, most preferably intravenous or subcutaneous injections, depending in part on whether the administration is brief or chronic.

The cells should be administered such that a therapeutic number resides in the body. The number of cells administered to an animal, particularly a human, in the context of the present invention should be sufficient to effect a therapeutic response in the animal over a reasonable period of time.

The modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. lmnmunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibodycoated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

The cells can be administered as part of an organ, tissue or cell (e.g. bone marrow) transplant.

The routes of administration and dosages described are intended only as a guide since a skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

The present invention will now be described by way of examples which are intended to be illustrative only and non-limiting:

### EXAMPLES

### Example 1

### Treatment of Patients Undergoing Bone Marrow Transplantation

Dendritic cells (DCs) are isolated from the transplant recipient by a suitable method (e.g. as described in U.S. Pat. No. 5,789,148) approximately 14 days prior to transplantation. They are maintained in culture in tissue culture medium such as RPMI-1640 supplemented with up to 10% autologous or ABO human serum. Inducers of Notch-ligand expression are added for the appropriate time (between 3 hours and 2 days). Cytokines such as IL-4 and GM-CSF are also added as required.

Donor individuals for the bone marrow transplantation procedure are selected from an appropriate category (live related, MHC-matched unrelated or unmatched). T-cells from the donor are obtained by an appropriate method (e.g. as described in U.S. Pat. No. 4,663,058) and may be enriched by standard methods including antibody-mediated separation.

The donor cells are cultured in RPMI-1640 with serum and in the presence of modified DCs.

The T-cells and DCs are then transferred to the transplant patient by infusion at a suitable time.

### Example 2

### Generation of Regulatory T-cells

We define regulatory T-cells as those that can transfer antigen specific suppression to other T-cells.

2.1—Mixed Lymphocyte Reactions Between Donors and Recipients with Varying Degrees of HLA Mismatch

Patient DCs are purified and cultured as set out in Example 1. They are transduced with Serrate 1 using retrovirus or non-viral protocols. Successfully transformed DCs are then co-cultured with donor peripheral blood mononuclear cells (PBMCs) in sufficient numbers.

2.2—Mixed Lymphocyte Reaction to Demonstrate the Induction of “Regulating” or Tolerising T-cells

Two experimental protocols were designed.

### Experimental Design A

Patient DCs are purified and cultured as set out in Example 1. They are transduced with Serrate 1 using retrovirus or non-viral protocols. Successfully transformed DCs are then co-cultured with donor T-cells in sufficient numbers.

The donor T-cells are then isolated and added to cultures containing irradiated recipient PMBCs (or a HLA mismatched control) and naive donor PMBCs.

Proliferation and cytokine production of the donor cells are measured and Notch 1 expression and cleavage is detected by Western Blotting.

### Experimental Design B

Host Antigen Presenting Cells are transduced with Serrate 1 and co-cultured with T-cell clones from the donor in the presence of a peptide to produce tolerised T-cells. These T-cells are then irradiated and co-cultured with naive T-cell clones and re-stimulated with the APC and peptide.

Proliferation and cytokine production of the donor cells are again measured and Notch 1 expression and cleavage is detected by Western Blotting.

2.3—Mouse Model of Bone Marrow Transplant in vitro and in vivo

Three experimental protocols were designed.

### Experimental Design A

DCs are purified from F1 mice and transduced with Serrate 1. The transduced cells are then co-cultured with parental T-cells in sufficient numbers.

Proliferation and cytokine production of the T-cells are measured and Notch 1 expression and cleavage is detected by Western Blotting.

### Experimental Design B

DCs are purified from F1 mice and transduced with Serrate 1. The transduced cells are co-cultured with parental T-cells which are then added to cultures containing naive T-cells and irradiated F1 stimulator cells.

Proliferation and cytokine production of the T-cells are measured and Notch 1 expression and cleavage is detected by Western Blotting.

### Experimental Design C

DCs are purified from F1 mice and transduced with Serrate 1. The transduced cells are then co-cultured with parental T-cells.

T-cell depleted bone marrow together with escalating doses of regulatory T-cells is injected into the F1 mouse. T-cell reconstitution and the ability to respond to other antigens are then measured.

### Example 3

### Preparation of MyOne or CELLection Dynal Microbeads

MyOne Streptavidin beads (1 μm, Dynal 650.01) and CELLection Biotin Binder (4.5 μm, Dynal 115.21) were coated with anti-hIgG4-Biotin antibodies based on the binding capacity recommended by the supplier.

Briefly, 20 μg of anti-hIgG4-biotin (BD Biosciences) were incubated 30 minutes at room temperature with either 1 mg of MyOne beads (equivalent to 7-12×10⁸ beads) or 10⁸ CELLection beads. The beads were then washed and further incubated with either 100 μg of human Delta1EC domain-hIgG4 fusion protein (prepared as described in WO 03/041735; Example 1) or 100 μg of hIgG4 (Sigrna) for 2 hours at room temperature. After washing, the MyOne beads and the CELLection beads were resuspended in 500 μl of RPMI/BSA 0.1% and stored at 4° C.

### Example 4

### Modulation of Cytokine Production by Delta1-hIgG4 Immobilised on MyOne or CELLection Dynal Microbeads During a Mixed Lymphocyte Reaction

Human peripheral blood mononuclear cells (PBMC) were purified from blood of 2 donors (donor A and donor B).

Human CD14+ monocytes and CD4+ T cells were isolated from PBMC from donor A and B respectively by positive selection using anti-CD14 and anti-CD4 microbeads from Miltenyi Biotech according to the manufacturer's instructions.

The CD14+ cells (donor A) were differentiated into dendritic cells (DC) by incubation for 6 days in medium [RPMI/10%FCS/glutamine/B2-mercaptoethanol/antibiotics] in the presence of hGM-CSF 50 ng/ml and hIL-4 50 ng/ml (both from Peprotech). Dendritic cell maturation was induced by addition into the culture of LPS 1 μg/ml (Sigma L-2654) for the last 24 hours.

Matured-DC were treated for 1 hour with 50 μg/ml Mitomycin C (Sigma) in RPMI and washed 4 times. These cells were then plated at 4×10⁴, 1×10⁴, 2.5×10³, 6.25×10² cells/well in triplicates in a 96-well-plate in RPMI medium containing 10% FCS, glutamine, penicillin, streptomycin and β₂-mercaptoethanol. 2×10⁵ Allogenic CD4+ T cells (donor B) were added into each well given a final volume of 200 μl/well.

10 μl of beads (Example 3) coated with human Delta1EC domain-hIgG4 fusion protein or control beads were added in some of the wells.

The supernatants were removed after 5 days of incubation at 37° C./5%CO₂/humidified atmosphere and cytokine production was evaluated by ELISA using Pharmingen kits OptElA Set human IL 10 (catalog No. 555157) and a human TNFa DuoSet from R&D Systems (catalog. No. DY210) for TNFa according to the manufacturer's instructions.

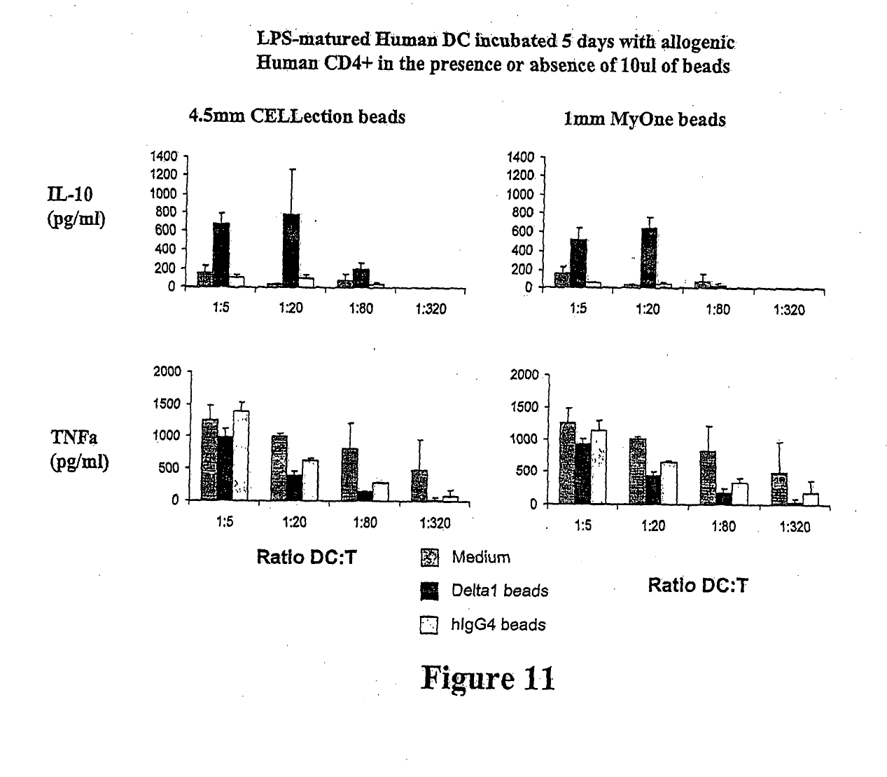

Results are shown in FIG. 11.

The invention is further described in the following numbered paragraphs.

1. Use of a modulator of Notch signalling for the preparation of a medicament for treatment of Graft Versus Host Disease (GVHD).

2. Use of a modulator of Notch signalling for the preparation of a medicament for treatment of diseases and conditions caused by or associated with an organ, tissue or cell transplant.

3. Use according to claim 2 for the preparation of a medicament for treatment of diseases and conditions caused by or associated with bone marrow transplants.

4. Use according to any one of claims 1-3 wherein the modulator is selected from the group consisting of: an organic compound, a inorganic compound, a peptide or polypeptide, a polynucleotide, an antibody, a fragment of an antibody, a cytokine and a fragment of a cytokine.

5. Use according to any preceding claim wherein the modulator is capable of activating and/or up-regulating Notch signalling.

6. Use according to any preceding claim wherein the modulator is capable of activating and/or up-regulating the expression and/or activity of at least one Notch ligand.

7. Use according to claim 6 wherein the modulator is a Notch ligand or a fragment or analogue thereof which retains the signalling transduction ability of Notch ligand, or a polynucleotide sequence which encodes therefor.

8. Use according to claim 7 wherein the modulator is derived from the Delta or Serrate family of proteins, or a polynucleotide sequence which encodes therefor.

9. Use according to claim 6 wherein the modulator is selected from a transcription factor from the achaete/scute complex, Follistatin, Xnr3, Noggin, Chordin, a fibroblast growth factor, an immunosuppressive cytokine and derivatives, fragments, variants and homologues thereof, or a polynucleotide which encodes therefor.

10. Use according to claim 9 wherein the immunosuppressive cytokine is selected from IL-4, IL-10, IL-13, TGF-β and SLIP3 ligand, or a polynucleotide which encodes therefor.

11. Use according to any one of claims 1-5 wherein the modulator is capable of down-regulating and/or inhibiting the expression and/or activity of an antagonist of Notch ligand expression or activity.

12. Use according to claim 11 wherein the antagonist is selected from a Bone Morphogenic Protein (BMP), a BMP receptor, activin, a Toll-like receptor (TLRs), Mesp2, a cytokine and derivatives, fragments, variants and homologues thereof.

13. Use according to claim 12 wherein the cytokine is selected from IL-12, IFN-γ and TFN-α.

14. Use according to claim 11 wherein the modulator is a polynucleotide sequence.

15. Use according to claim 14 wherein the polynucleotide sequence is or encodes an antisense sequence.

16. Use according to claim 15 wherein the antisense sequence is derived from a sense nucleotide sequence encoding a polypeptide selected from a Bone Morphogenic Protein (BMP), a BMP receptor, activin, a Toll-like receptor (TLRs), Mesp2, a cytokine and derivatives, fragments, variants and homologues thereof.

17. Use according to claim 16 wherein the cytokine is selected from IL-12, IFN-γ and TFN-α.

18. Use according to any preceding claim wherein preparation of the medicament comprises:
(i) isolating an antigen presenting cell (APC) from a transplant patient;
(ii) exposing the cell to the modulator; and
(iii) incubating said cell with APCs or lymphocytes from a transplant donor.

19. Use according to claim 18 wherein step (ii) comprises bringing the APC from a transplant patient into direct contact with the modulator thereby causing activation and/or up-regulation of the expression and/or activity of at least one Notch ligand in the APC.

20. Use according to claim 18 wherein step (ii) comprises transforming the APC from a transplant patient with the modulator or a polynucleotide sequence encoding the modulator thereby causing activation and/or up-regulation of the expression and/or activity of at least one Notch ligand in the APC.

21. Use according to any one of claims 18-20 wherein the APC of step (i) is a dendritic cell (DC).

22. Use according to any one of claims 18-21 wherein the APCs or lymphocytes of step (iii) are T-cells.

23. Use according to any one of claims 1-5 wherein the modulator is capable of activating and/or upregulating the expression and/or activity of Notch.

24. Use according to claim 23 wherein the modulator is a Notch ligand or a fragment or analogue thereof which retains the signalling transduction ability of Notch ligand, or a polynucleotide sequence which encodes therefor.

25. Use according to claim 24 wherein the modulator is derived from the Delta or Serrate family of proteins.

26. Use according to claim 23 wherein the modulator is selected from Notch and a derivative, fragment, variant or homologue thereof, or a polynucleotide sequence encoding therefor.

27. Use according to claim 26 wherein the modulator is a constitutively active form of Notch.

28. Use according to any one of claims 23-27 wherein preparation of the medicament comprises:
(i) isolating an APC or lymphocyte from a transplant donor;
(ii) exposing the APC or lymphocyte to the modulator; and
(iii) incubating said cell with APCs from a transplant patient.

29. Use according to claim 28 wherein step (ii) comprises bringing the APC or lymphocyte from a transplant donor into direct contact with the modulator thereby causing activation and/or up-regulation of the expression and/or activity of Notch in the APC or lymphocyte.

30. Use according to claim 28 wherein step (ii) comprises transforming the APC or lymphocyte from a transplant donor with the modulator or a polynucleotide sequence encoding the modulator thereby causing activation and/or up-regulation of the expression and/or activity of Notch in the APC or lymphocyte.

31. Use according to any one of claims 28-30 wherein the APC or lymphocyte of step (i) is a T-cell.

32. Use according to any one of claims 28-31 wherein the APCs of step (iii) are dendritic cells (DCs).

33. Use according to any one of claims 1 and 4-32 for the treatment of infection caused by immuno-suppression, inflammatory reactions and diseases, erythroderma, severe blistering, gastrointestinal haemorrhage, flilminant liver failure, jaundice, scleroderma, joint contractures, skin ulcers, erythematous macules, erythema, esophageal dysmotility, fevers, anthema, diarrhoea, vomituration, anorexia, abdominal pains, hepatopathy, hepatic insufficiency, hair loss and/or generalised wasting syndrome.

34. Use according to any one of claims 2-32 for the treatment of GVHD, infections caused by immuno-suppression, leukaemia, aplastic anaemia, thalassemia major, immunodeficiency diseases, multiple myelomas and/or lymphomas.

35. A method of preparing donor cells for use in a transplant comprising:
(i) isolating an antigen presenting cell (APC) from a transplant patient;
(ii) exposing the cell to a modulator of Notch signalling; and
(iii) incubating said cell with APCs or lymphocytes from the transplant donor.

36. A method according to claim 35 wherein the modulator is selected from the group consisting of: an organic compound, a inorganic compound, a peptide or polypeptide, a polynucleotide, an antibody, a fragment of an antibody, a cytokine and a fragment of a cytokine.

37. A method according to claim 36 wherein the modulator is capable of up-regulating Notch signalling.

38. A method according to claim 37 wherein the modulator is capable of activating and/or up-regulating the expression and/or activity of at least one Notch ligand.

39. A method according to claim 38 wherein the modulator is a Notch ligand or a fragment or analogue thereof which retains the signalling transduction ability of Notch ligand, or a polynucleotide sequence which encodes therefor.

40. A method according to claim 39 wherein the modulator is derived from the Delta or Serrate family of proteins, or a polynucleotide sequence which encodes therefor.

41. A method according to claim 38 wherein the modulator is selected from a transcription factor from the achaete/scute complex, Follistatin, Xnr3, Noggin, Chordin, a fibroblast growth factor, an immunosuppressive cytokine and derivatives, fragments, variants and homologues thereof, or a polynucleotide which encodes therefor.

42. A method according to claim 41 wherein the immunosuppressive cytokine is selected from IL-4, IL-10, IL-13, TGF-β and SLIP3 ligand, or a polynucleotide which encodes therefor.

43. A method according to claim 37 wherein the modulator is capable of down-regulating and/or inhibiting the expression and/or activity of an antagonist of Notch ligand expression or activity.

44. A method according to claim 43 wherein the antagonist is selected from a Bone Morphogenic Protein (BMP), a BMP receptor, activin, a Toll-like receptor (TLRs), Mesp2, a cytokine and derivatives, fragments, variants and homologues thereof.

45. A method according to claim 44 wherein the cytokine is selected from IL-12, IFN-γ and TFN-α.

46. A method according to claim 43 wherein the modulator is a polynucleotide sequence.

47. A method according to claim 46 wherein the polynucleotide sequence is or encodes an antisense sequence.

48. A method according to claim 47 wherein the antisense sequence is derived from a sense nucleotide sequence encoding a polypeptide selected from a Bone Morphogenic Protein (BMP), a BMP receptor, activin, a Toll-like receptor (TLRs), Mesp2, a cytokine and derivatives, fragments, variants and homologues thereof.

49. A method according to claim 48 wherein the cytokine is selected from IL-12, IFN-γ and TFN-α.

50. A method according to any one of claims 35-49 wherein the APC of step (i) is a dendritic cell (DC).

51. A method according to any one of claims 35-50 wherein the APCs or lymphocytes of step (iii) are T-cells.

52. A method of preparing donor cells for use in a transplant comprising:
(i) isolating an APC or lymphocyte from a transplant donor;
(ii) exposing the APC or lymphocyte to a modulator of Notch signalling; and
(iii) incubating said cell with APCs from a transplant patient.

53. A method according to claim 52 wherein the modulator is selected from the group consisting of: an organic compound, a inorganic compound, a peptide or polypeptide, a polynucleotide, an antibody, a fragment of an antibody, a cytokine and a fragment of a cytokine.

54. A method according to claim 53 wherein the modulator is capable of up-regulating Notch signalling.

55. A method according to claim 54 wherein the modulator is capable of activating and/or upregulating the expression and/or activity of Notch.

56. A method according to claim 55 wherein the modulator is a Notch ligand or a fragment or analogue thereof which retains the signalling transduction ability of Notch ligand, or a polynucleotide sequence which encodes therefor.

57. A method according to claim 56 wherein the modulator is derived from the Delta or Serrate family of proteins.

58. A method according to claim 55 wherein the modulator is selected from Notch and a derivative, fragment, variant or homologue thereof, or a polynucleotide sequence encoding therefor.

59. A method according to claim 58 wherein the modulator is a constitutively active form of Notch. 60. A method according to any one of claims 52-59 wherein step (ii) comprises bringing the APC or lymphocyte from a transplant donor into direct contact with the modulator thereby causing activation and/or up-regulation of the expression and/or activity of Notch in the APC or lymphocyte.

61. A method according to any one of claims 52-59 wherein step (ii) comprises transforming the APC or lymphocyte from a transplant donor with the modulator or a polynucleotide sequence encoding the modulator thereby causing activation and/or up-regulation of the expression and/or activity of Notch in the APC or lymphocyte.

62. A method according to any one of claims 52-61 wherein the APC or lymphocyte of step (i) is a T-cell.

63. A method according to any one of claims 52-62 wherein the APCs of step (iii) are dendritic cells (DCs).

64. A method of preparing donor cells according to any one of claims 35-63 for use in an organ, tissue or cell transplant.

65. A method according to claim 64 wherein the cell transplant is a bone marrow transplant.

66. Donor cell prepared according to the method of any one of claims 35-63.

67. Use of a donor cell according to claim 66 for the preparation of a medicament for treatment of GVHD.

68. Use according to claim 67 for the preparation of a medicament for treatment of infection caused by immuno-suppression, inflammatory reactions and/or diseases, erythroderma, severe blistering, gastrointestinal haemorrhage, fulminant liver failure, jaundice, scleroderma, joint contractures, skin ulcers, erythematous macules, erythema, esophageal dysmotility, fevers, anthema, diarrhoea, vomituration, anoraxia, abdominal pain, hepatopathy, hepatic insufficiency, hair loss and/or generalised wasting syndrome.

69. Use of a donor cell according to claim 66 for the preparation of a medicament for treatment of diseases and conditions caused by or associated with an organ, tissue or cell transplant.

70. Use according to claim 69 for the preparation of a medicament for treatment of diseases and conditions caused by or associated with a bone marrow transplant.

71. Use according to claim 70 for the preparation of a medicament for treatment of GVHD, infections caused by immuno-suppression, leukaemia, aplastic anaemia, thalassemia major, immunodeficiency diseases, multiple myelomas and/or lymphomas.

72. A pharmaceutical composition for use in the treatment of GVHD comprising donor cells according to claim 66 together with a pharmaceutically acceptable carrier.

73. A pharmaceutical composition according to claim 72 for use in the treatment of infection caused by immuno-suppression, inflammatory reactions, erythroderma, severe blistering, gastrointestinal haemorrhage, fulminant liver failure, jaundice, scleroderma, joint contractures, skin ulcers, erythematous macules, erythema, esophageal dysmotility, fevers, anthema, diarrhoea, vomituration, anoraxia, abdominal pain, hepatopathy, hepatic insufficiency, hair loss and/or generalised wasting syndrome.

74. A pharmaceutical composition for use in the treatment of diseases and conditions caused by or associated with an organ, tissue or cell transplant comprising donor cells according to claim 66 together with a pharmaceutically acceptable carrier.

75. A pharmaceutical composition according to claim 74 for use in the treatment of diseases and conditions caused by or associated with a bone marrow transplant.

76. A pharmaceutical composition according to claim 75 for use in the treatment of GVHD, infections caused by immuno-suppression, leukaemia, aplastic anaemia, thalassemia major, immunodeficiency diseases, multiple myelomas and/or lymphomas.

### REFERENCES

Ausubel et al. (1995) Current Protocols in Molecular Biology, John Wiley & Sons, Inc.
Caux C, et al. (1992) Nature 360: 258-261
Coffin R S, et al. (1998) Gene Therapy 5: 718-722
Cosulich et al. (1987) PNAS 84: 4205
Dkuz et al. (1997) Cell 90:271-280
Gennaro, A. (1985) Remington's Pharmaceutical Sciences, Mack Publishing Co
Guatelli et al. (1990) Proc. Natl. Acad. Sci. USA 87:1874
Hara et al. (1986) J. Exp. Med. 164: 1988
Harlow and Lane (1988) Antibodies: a Laboratory Manual, Cold Spring Harbour
Hemmati-Brivanlou and Melton (1997) Cell 88: 13-17
Hoyne, G. etal. (1999) Int Immunology 12(2):177-185
Hoyne, G. et al. (1999) Int Arch Allergy Immunol 118:122-124
Hoyne, G. et al. (2000) Immunology 100:281-288
Hoyne, G. etal. (2001) Immunological Reviews 182:215-227
Iemura et al. (1998) PNAS 95: 9337-9342
Inaba K, et al. (1992) J. Exp. Med. 175: 1157-1167
Kohler and Milstein (1975) Nature 256: 495-497
Lamb et al. (1983) J. Exp. Med. 157: 1434-1447
Landegren, U. et al. (1988) Science 242:229-237
Lee et al. (2000) Curr. Biol. 10(15): 931-4
Lewis, C. and McGee, J. (1992) The Natural Killer Cell, Oxford University Press
Lewis, R. (1990) Genetic Engin. News 10(1): 54-55
Li et al. (1998) Immunity 8(1):43-55
Lizardi et al. (1998) Nat Genet 19: 225
Medhzhitov et al. (1997) Nature 388: 394-397
Melton et al. Nuc. Acids Res. 12: 7035
Mak, T. and Simard, J. (1998) Ch. 7, Handbook of Immune Response Genes
Mok et al. (1994) Gynaecologic Oncology 52: 247-252
Radbruch, ed. (1992) Flow Cytometry and Cell Sorting: A Laboratory Manual, Springer Laboratory, New York
Sallusto F and Lanzavecchia A (1994) J. Exp. Med. 179: 1109-1118
Sambrook et al. eds. (1989) Molecular Cloning: A Laboratory Manual, Cold Spring Harbour Laboratory Press, New York
Sasai et al. (1994) Cell 79: 779-790
Takahashi et al. (2000) Nat. Genet. 25(4): 390-6
Trinchieri, G. (1989) Adv. Immunol. 47: 187-376
Valenzuela et al. (1995) J. Neurosci 15: 6077-6084
Walker et al. (1992) PNAS (USA) 80: 392
Wilson and Hemmati-Brivanlou (1997) Neuron 18: 699-710
Wu, D. and Wallace, R. (1989) Genomics 4: 560
Zhao et al. (1995) J. Immunol 155:3904-3911.

### ***

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

We claim:

1. A method of treating Graft Versus Host Disease (GVHD) in a subject comprising administering a modulator of Notch signalling to the subject.

2. The method according to claim 1, wherein GVHD is caused by or associated with an organ, tissue or cell transplant.

3. The method according to claim 1, wherein the modulator is selected from the group consisting of: an organic compound, a inorganic compound, a peptide or polypeptide, a polynucleotide, an antibody, or a fragment of an antibody.

4. The method according to claim 3, wherein the modulator is a Notch ligand or a fragment or analogue thereof, which retains the signalling transduction ability of Notch ligand, or a polynucleotide sequence which encodes therefor.

5. The method according to claim 4, wherein the modulator is derived from the Delta or Serrate family of proteins, or a polynucleotide sequence which encodes therefor.

6. The method according to claim 3, wherein the modulator is selected from Notch and a derivative, fragment, variant or homologue thereof, or a polynucleotide sequence encoding therefor.

7. The method according to claim 6, wherein the modulator is a constitutively active form of Notch.

8. A method of preparing donor cells for use in a transplant comprising:
(i) isolating an antigen presenting cell (APC) from a transplant patient;
(ii) exposing the APC to a modulator of Notch signalling; and
(iii) incubating the APC with APCs or lymphocytes from a transplant donor.

9. The method according to claim 8, wherein step (ii) comprises bringing the APC from the transplant patient into direct contact with the modulator, thereby causing activation and/or up-regulation of the expression and/or activity of at least one Notch ligand in the APC.

10. The method according to claim 8, wherein step (ii) comprises transforming the APC from the transplant patient with the modulator or a polynucleotide sequence encoding the modulator, thereby causing activation and/or up-regulation of the expression and/or activity of at least one Notch ligand in the APC.

11. The method according to claim 8, wherein the APC from the transplant patient is a dendritic cell (DC).

12. The method according to claim 8, wherein the APCs or lymphocytes from the transplant donor are T-cells.

13. A method of preparing donor cells for use in a transplant comprising:
(i) isolating an APC or lymphocyte from a transplant donor;
(ii) exposing the APC or lymphocyte to a modulator of Notch signalling; and
(iii) incubating said APC or lymphocyte with APCs from a transplant patient.

14. The method according to claim 13, wherein step (ii) comprises bringing the APC or lymphocyte from the transplant donor into direct contact with the modulator, thereby causing activation and/or up-regulation of the expression and/or activity of Notch in the APC or lymphocyte.

15. The method according to claim 13, wherein step (ii) comprises transforming the APC or lymphocyte from the transplant donor with the modulator or a polynucleotide sequence encoding the modulator, thereby causing activation and/or up-regulation of the expression and/or activity of Notch in the APC or lymphocyte.

16. The method according to claim 13, wherein the APC or lymphocyte from the transplant donor is a T-cell.

17. The method according to claim 13, wherein the APCs from the transplant patient are dendritic cells (DCs).
